(19) United States
(12) Patent Application Publication
(10) Pub. No.: US 2025/0075261 A1
(43) Pub. Date: Mar. 6, 2025

(54) SPATIAL ARRAYS CONTAINING CAPTURE PROBES WITH DIFFERENT RELEASE MECHANISMS

(71) Applicant: 10x Genomics, Inc., Pleasanton, CA (US)

(72) Inventor: William Won Shik Kim, Los Altos Hills, CA (US)

(21) Appl. No.: 18/816,947

(22) Filed: Aug. 27, 2024

Related U.S. Application Data
(60) Provisional application No. 63/579,161, filed on Aug. 28, 2023.

(51) Int. Cl. C12Q 1/6837

(52) U.S. Cl. CPC C12Q 1/6837

(57) ABSTRACT

The present disclosure features methods, composition, and kits including spatial arrays including capture probes with two or more different release mechanisms.

### CROSS-REFERENCE TO RELATED APPLICATIONS

This application claims the benefit of U.S. Provisional Patent Application No. 63/579,161, filed on Aug. 28, 2023, which is incorporated herein by reference in its entirety.

### BACKGROUND

Cells within a tissue of a subject have differences in cell morphology and/or function due to varied analyte levels (e.g., gene and/or protein expression) within the different cells. The specific position of a cell within a tissue (e.g., the cell's position relative to neighboring cells or the cell's position relative to the tissue microenvironment) can affect, e.g., the cell's morphology, differentiation, fate, viability, proliferation, behavior, signaling and cross-talk with other cells in the tissue.

Spatial heterogeneity has been previously studied using techniques that only provide data for a small handful of analytes in the context of an intact tissue or a portion of a tissue, or provides substantial analyte data for dissociated tissue (i.e., single cells), but fail to provide information regarding the position of the single cell in a parent biological sample (e.g., tissue sample).

Expenses associated with spatial arrays include the cost of consumables such as spatial arrays and reagents, as well as sequencing costs. The present disclosure features spatial arrays including capture probes with different release mechanisms (e.g., restriction sites, cleavage domains, photocleavable domains, etc.) that allow re-use or multiplexed use of the spatial array. Reusing the array provides for capturing one or more different analytes from the same biological sample and/or capturing analytes from different biological samples (e.g., a second biological sample, a third biological sample, a fourth biological sample, or more).

### SUMMARY

In general, the quantity of spatially-barcoded capture probes on an array outnumber the quantity of analytes detected in a given biological sample. The present disclosure features methods, compositions, and kits that include different sets of capture probes (e.g., a first set, a second set, a third set, or more) on the same array. The sets of capture probes can differ from each other in various ways. For example, the sets of capture probes can have different release mechanisms (e.g., restriction sites, cleavage domains, photocleavable domains, etc.). Additionally, the sets of capture probes can have different capture domains.

Thus, provided herein are methods for determining the spatial location of an analyte in a biological sample, the method including: (a) disposing the biological onto a first substrate; (b) providing a second substrate including an array, where the array includes a first set of capture probes and a second set of capture probes, where a first capture probe of the first set of capture probes includes: (i) a first spatial barcode; (ii) a first capture domain; and (iii) a first restriction endonuclease cleavage site; and where a second capture probe of the second set of capture probes includes: (i) a second spatial barcode; (ii) a second capture domain; and (iii) a second restriction endonuclease cleavage site; where the sequence of the first restriction endonuclease cleavage site of the first capture probe differs from the sequence of the second restriction endonuclease cleavage site of the second capture probe; (c) hybridizing the analyte to the first capture domain; (d) releasing the first set of capture probes from the array; and (c) determining (i) all or a part of the sequence of the analyte, or a complement thereof, and (ii) the sequence of the first spatial barcode, or a complement thereof, and using the determined sequences of (i) and (ii) to determine the spatial location of the analyte in the biological sample.

In some embodiments, the first restriction endonuclease cleavage site is 5′ to the first spatial barcode and the first capture domain of the first capture probe, and/or where and the second restriction endonuclease cleavage site is 5′ to the second spatial barcode and the second capture domain of the second capture probe.

In some embodiments, the sequence of the first restriction endonuclease cleavage site and/or the sequence of the second restriction endonuclease site is about 6-12 nucleotides in length, preferably about 7-8 nucleotides in length.

In some embodiments, the releasing of the first set of capture probes includes contacting the array with a first restriction endonuclease.

Also provided herein are methods for determining the spatial location of an analyte in a biological sample, the method including: (a) disposing the biological onto a first substrate; (b) providing a second substrate including an array, where the array includes a first set of capture probes and a second set of capture probes, where a first capture probe of the first set of capture probes includes: (i) a first spatial barcode; (ii) a first capture domain; and (iii) a first photocleavable cleavage site; and where a second capture probe of the second set of capture probes includes: (i) a second spatial barcode; (ii) a second capture domain; and (iii) a second photocleavable cleavage site; where the first photocleavable cleavage site of the first capture probe differs from the second photocleavable cleavage site of the second capture probe; (c) hybridizing the analyte to the first capture domain; (d) releasing the first set of capture probes from the array; and (c) determining (i) all or a part of the sequence of the analyte, or a complement thereof, and (ii) the sequence of the first spatial barcode, or a complement thereof, and using the determined sequences of (i) and (ii) to determine the spatial location of the analyte in the biological sample.

In some embodiments, the first photocleavable cleavage site is 5′ to the first spatial barcode and the first capture domain of the first capture probe, and where and/or the second photocleavable cleavage site is 5′ to the second spatial barcode and the second capture domain of the second capture probe.

In some embodiments, the first photocleavable cleavage site and the second photocleavable cleavage site include one or more nucleosides including a photoreactive group. In some embodiments, the photoreactive group is selected from o-Nitrobenzyl (o-NB) derivatives, p-Hydroxyphenacyl, TEEP-OH, Aryl sulfide, and nitroindole.

In some embodiments, the releasing of the first set of capture probes includes exposing the array to light. In some embodiments, the method includes releasing the second set of capture probes, where the first photocleavable cleavage site and the second photocleavable cleavage site are cleaved by exposure to different wavelengths of light.

In some embodiments, releasing of the first set of capture probes is irreversible.

In some embodiments, releasing the first set of capture probes from the array is performed after the analyte hybridizes to the first capture domain. In some embodiments, hybridizing the analyte to the first capture domain is performed after releasing the first set of capture probes from the array.

In some embodiments, the method includes hybridizing a second analyte in the biological sample or an analyte from a second biological sample to the second capture domain. In some embodiments, hybridizing the second analyte to the second capture domain is performed before releasing the second set of capture probes from the array. In some embodiments, hybridizing the second analyte to the second capture domain is performed after releasing the second set of capture probes from the array.

In some embodiments, the array includes one or more additional sets of capture probes and where the one or more additional sets of capture probes each have a different release mechanism than the first and second set of capture probes. In some embodiments, the method includes hybridizing one or more additional analytes in the biological sample or in different biological samples to a capture probe in the one or more additional sets of capture probes, and releasing the one or more additional sets of capture probes from the array.

Also provided herein are methods for determining the spatial location of an analyte in a biological sample, the method including: (a) disposing the biological onto a first substrate; (b) hybridizing a first probe and a second probe to the analyte, where the first probe and the second probe each include a sequence that is substantially complementary to sequences of the analyte, and where the second probe includes a capture probe binding domain; (c) coupling the first probe and the second probe, thereby generating a ligation product; (d) providing a second substrate including an array, where the array includes a first set of capture probes and a second set of capture probes, where a first capture probe of the first set of capture probes includes: (i) a first spatial barcode; (ii) a first capture domain; and (iii) a first restriction endonuclease cleavage site; and where a second capture probe of the second set of capture probes includes: (i) a second spatial barcode; (ii) a second capture domain; and (iii) a second restriction endonuclease cleavage site; where the sequence of the first restriction endonuclease cleavage site of the first capture probe differs from the sequence of the second restriction endonuclease cleavage site of the second capture probe; (c) hybridizing the ligation product to the first capture domain of the first capture probe; (f) releasing the first set of capture probes from the array; and (g) determining (i) all or a part of the sequence of the analyte, or a complement thereof, and (ii) the sequence of the first spatial barcode, or a complement thereof, and using the determined sequences of (i) and (ii) to determine the spatial location of the analyte in the biological sample.

In some embodiments, the first restriction endonuclease cleavage site is 5′ to the first spatial barcode and the first capture domain of the first capture probe, and where and the second restriction endonuclease cleavage site is 5′ to the second spatial barcode and the second capture domain of the second capture probe.

In some embodiments, the sequence of the first restriction endonuclease cleavage site and/or the sequence of the second restriction endonuclease site is about 6-12 nucleotides in length, preferably about 7-8 nucleotides in length.

In some embodiments, releasing of the first set of capture probes includes contacting the array with a first restriction endonuclease.

Also provided herein are methods for determining the spatial location of an analyte in a biological sample, the method including: (a) disposing the biological sample onto a first substrate; (b) hybridizing a first probe and a second probe to the analyte, where the first probe and the second probe each include a sequence that is substantially complementary to adjacent sequences of the analyte, and where the second probe includes a capture probe binding domain; (c) coupling the first probe and the second probe, thereby generating a ligation product; (d) providing a second substrate including an array, where the array includes a first set of capture probes and a second set of capture probes, where a first capture probe of the first set of capture probes includes: (i) a first spatial barcode; (ii) a first capture domain; and (iii) a first photocleavable cleavage site; and where a second capture probe of the second set of capture probes includes: (i) a second spatial barcode; (ii) a second capture domain; and (iii) a second photocleavable cleavage site; where the first photocleavable site of the first capture probe differs from the second photocleavable site of the second capture probe; (c) hybridizing the ligation product to the first capture domain of the first capture probe; (f) releasing the first set of capture probes from the array; and (g) determining (i) all or a part of the sequence of the analyte, or a complement thereof, and (ii) the sequence of the first spatial barcode, or a complement thereof, and using the determined sequences of (i) and (ii) to determine the spatial location of the analyte in the biological sample.

In some embodiments, the first photocleavable cleavage site is 5′ to the first spatial barcode and the first capture domain of the first capture probe, and where and the second photocleavable cleavage site is 5′ to the second spatial barcode and the second capture domain of the second capture probe.

In some embodiments, the first photocleavable cleavage site and the second photocleavable cleavage site include one or more nucleosides including a photoreactive group. In some embodiments, the photoreactive group is selected from o-Nitrobenzyl (o-NB) derivatives, p-Hydroxyphenacyl, TEEP-OH, Aryl sulfide, and nitroindole.

In some embodiments, releasing of the first set of capture probes includes exposing the array to light. In some embodiments, releasing of the first set of capture probes is irreversible.

In some embodiments, releasing the first set of capture probes from the array is performed after the ligation product hybridizes to the first capture domain. In some embodiments, hybridizing the ligation product to the first capture domain is performed after releasing the first set of capture probes from the array. In some embodiments, the method includes hybridizing a ligation product from a second analyte or a ligation product from a second biological sample to the second capture domain.

In some embodiments, hybridizing the ligation product from the second analyte or the ligation product from a second biological sample to the second capture domain is performed before releasing the second set of capture probes from the array. In some embodiments, hybridizing the ligation product from the second analyte or the ligation product from a second biological sample to the second capture domain is performed after releasing the second set of capture probes from the array.

In some embodiments, the first photocleavable cleavage site and the second photocleavable cleavage site are cleaved by exposure to different wavelengths of light.

In some embodiments, the array includes one or more additional sets of capture probes. In some embodiments, the one or more additional sets of capture probes each have a different release mechanism than the first and second set of capture probes.

In some embodiments, the method includes hybridizing one or more additional ligation products from one or more additional analytes to a capture probe in the one or more additional sets of capture probes, and releasing the one or more additional sets of capture probes from the array.

In some embodiments, the method includes releasing the ligation product from the analyte. In some embodiments, releasing includes the use of an RNase.

In some embodiments, the method includes extending the first capture probe using the analyte as a template. In some embodiments, the method includes extending the first capture probe using the ligation product as a template.

In some embodiments, at least a portion of the biological sample is aligned with at least a portion of the array.

In some embodiments, the sequence of the first spatial barcode and the sequence of the second spatial barcode are each unique to a distinct position on the array. In some embodiments, the first spatial barcode and the second spatial barcode include the same sequence. In some embodiments, the first spatial barcode and the second spatial barcode include different sequences.

In some embodiments, the first capture domain and the second capture domain include different sequences. In some embodiments, the first capture domain and the second capture domain include the same sequence.

In some embodiments, coupling includes ligating the first probe and the second probe via a ligase. In some embodiments, the ligase is one or more of a T4 RNA ligase (Rnl2), a PBCV-1 DNA ligase, a Chorella virus DNA ligase, a single-stranded DNA ligase, or a T4 DNA ligase.

In some embodiments, the first probe and/or the second probe includes a primer sequence.

In some embodiments, hybridizing the first probe and the second probe to the analyte includes contacting the biological sample with about 5000 or more probe pairs collectively including the first probe and the second probe. In some embodiments, the first probe and/or the second probe is a DNA probe.

In some embodiments, the method includes releasing the ligation product from the analyte, where releasing includes contacting the ligation product with an endoribonuclease. In some embodiments, the endoribonuclease is an RNase H enzyme.

In some embodiments, the first probe and the second probe are substantially complementary to adjacent sequences of the analyte. In some embodiments, the first probe and the second probe hybridize to sequences that are not adjacent to each other on the analyte. In some embodiments, the first probe is extended with a DNA polymerase, thereby (i) filling in a gap between the first probe and the second probe and (ii) generating an extended first probe.

In some embodiments, the analyte is a nucleic acid. In some embodiments, the nucleic acid is an RNA molecule. In some embodiments, the RNA molecule is mRNA. In some embodiments, the nucleic acid is a DNA molecule. In some embodiments, the DNA molecule is genomic DNA.

In some embodiments, the method includes contacting the biological sample with one or more analyte capture agents, where an analyte capture agent includes: (i) an analyte binding moiety; and (ii) an oligonucleotide including an analyte binding moiety barcode and an analyte capture sequence.

In some embodiments, the analyte binding moiety barcode identifies the analyte. In some embodiments, the analyte is a protein.

In some embodiments, the analyte capture sequence hybridizes to the second capture domain of the second capture probe.

In some embodiments, the analyte binding moiety is an antibody or an antigen binding fragment thereof.

In some embodiments, the method includes i) extending the second capture probe using the oligonucleotide as a template, thereby generating an extended capture probe including the second spatial barcode the analyte binding moiety barcode or a complement thereof, optionally, a unique molecular identifier (UMI) of the second capture probe, and optionally an analyte capture sequence or a complement thereof; and/or ii) extending the oligonucleotide using the second capture probe as a template, thereby generating an extended oligonucleotide.

In some embodiments, the method includes amplifying all or a portion of the extended capture probe, or a complement thereof.

In some embodiments, the first substrate and/or the second substrate comprises a glass slide.

In some embodiments, the method includes aligning the biological sample to the array where the aligning includes: mounting the first substrate on a first member of a support device, the first member configured to retain the first substrate; mounting the second substrate on a second member of the support device; applying a reagent medium to the first substrate and/or the second substrate; and operating an alignment mechanism of the support device to move the first member and/or the second member such that at least a portion of the biological sample is aligned with at least a portion of the array, and such that the portion of the biological sample and the portion of the array contact the reagent medium. In some embodiments, the alignment mechanism is coupled to the first member, the second member, or both the first member and the second member. In some embodiments, the alignment mechanism includes a linear actuator, optionally where: the linear actuator is configured to move the second member along an axis orthogonal to the plane of the first member and/or the second member, and/or the linear actuator is configured to move the first member along an axis orthogonal to the plane of the first member and/or the second member, and/or the linear actuator is configured to move the first member, the second member, or both the first member and the second member at a velocity of at least 0.1 mm/sec, and/or the linear actuator is configured to move the first member, the second member, or both the first member and the second member with an amount of force of at least 0.1 lbs.

In some embodiments, a separation distance is maintained between the first substrate and the second substrate, optionally where the separation distance is less than 50 microns, optionally where the separation distance is between 2-25 microns.

In some embodiments, the first substrate and/or the second substrate are moved via the alignment mechanism, the first substrate is at an angle relative to the second substrate such that a dropped side of the first substrate and a portion of the second substrate contact the reagent medium, optionally where: the dropped side of the first substrate urges the reagent medium toward the opposite direction, and/or the alignment mechanism moves the first substrate and/or the second substrate to maintain an approximately parallel arrangement of the first substrate and the second substrate and a separation distance between the first substrate and the second substrate, optionally when the approximately parallel arrangement and the separation distance are maintained, the spacer fully encloses and surrounds the at least portion of the biological sample and the at least portion of the array, and the spacer forms the sides of the chamber which hold a volume of the reagent medium.

In some embodiments, the first capture probe and/or the second capture probe includes one or more functional domains, a unique molecular identifier, a cleavage domain, or combinations thereof. In some embodiments, the one or more functional domains include a sequencing site or a primer binding site.

In some embodiments, the determining step includes sequencing. In some embodiments, the sequencing includes high-throughput sequencing.

In some embodiments, the biological sample is a tissue sample. In some embodiments, the tissue sample is a fixed tissue sample or a fresh-frozen tissue sample. In some embodiments, the biological sample is a tissue section. In some embodiments, the tissue section is a fresh-frozen tissue section. In some embodiments, the biological sample is a fixed tissue section. In some embodiments, the fixed tissue sample is a formalin-fixed paraffin-embedded (FFPE) tissue section, a paraformaldehyde-fixed tissue section, an acetone-fixed tissue section, a methanol-fixed tissue section, or an ethanol-fixed tissue section.

In some embodiments, the biological sample is fixed prior to or after mounting on the first substrate. In some embodiments, the method includes staining the biological sample. In some embodiments, the staining includes hematoxylin and/or cosin staining. In some embodiments, the staining includes the use of a detectable label selected from the group consisting of a radioisotope, a fluorophore, a chemiluminescent compound, a bioluminescent compound, or a combination thereof.

In some embodiments, the method includes determining the spatial location of one or more additional analytes in the biological sample.

In some embodiments, the method includes imaging the biological sample. In some embodiments, the imaging includes one or more of light field, bright field, dark field, phase contrast, fluorescence microscopy, reflection, interference, and confocal microscopy.

Also provided herein are kits including: an array including a first set of capture probes and a second set of capture probes, where a first capture probe of the first set of capture probes includes: (i) a first spatial barcode; (ii) a first capture domain; and (iii) a first restriction endonuclease cleavage site; and where a second capture probe of the second set of capture probes includes: (i) a second spatial barcode; (ii) a second capture domain; and (iii) a second restriction endonuclease cleavage site; where the sequence of the first restriction endonuclease cleavage site of the first capture probe differs from the sequence of the second restriction endonuclease cleavage site of the second capture probe.

Also provided herein are kits including: an array including a first set of capture probes and a second set of capture probes, where a first capture probe of the first set of capture probes includes: (i) a first spatial barcode; (ii) a first capture domain; and (iii) a first photocleavable cleavage site; and where a second capture probe of the second set of capture probes includes: (i) a second spatial barcode; (ii) a second capture domain; and (iii) a second photocleavable cleavage site; where the first photocleavable cleavage site of the first capture probe differs from the second photocleavable cleavage site of the second capture probe.

In some embodiments, the kit includes a plurality of first probes and a plurality of second probes, where a first probe of the plurality of first probes and a second probe of the plurality of second probes each include a sequence that is substantially complementary to sequences of an analyte, and where the second probe includes a capture probe binding domain.

In some embodiments, the kit includes a plurality of analyte capture agents, where an analyte capture agent includes: (i) an analyte binding moiety; (ii) an oligonucleotide including an analyte binding moiety barcode and a second analyte capture sequence.

In some embodiments, the kit includes one or more permeabilization reagents selected from protease, pepsin, proteinase K, and collagenase.

In some embodiments, the kit includes one or more of a ligase, a DNA polymerase, a reverse transcriptase, one or more crosslinking agents, one or more decrosslinking agents, and instructions for performing any of the methods described herein.

Also provided herein are compositions, including: an array including a first set of capture probes and a second set of capture probes, where a first capture probe of the first set of capture probes includes: (i) a first spatial barcode; (ii) a first capture domain; and (iii) a first restriction endonuclease cleavage site; and where a second capture probe of the second set of capture probes includes: (i) a second spatial barcode; (ii) a second capture domain; (iii) a second restriction endonuclease cleavage site; where the sequence of the first restriction endonuclease cleavage site of the first capture probe differs from the sequence of the second restriction endonuclease cleavage site of the second capture probe.

Also provided herein are compositions, including: an array including a first set of capture probes and a second set of capture probes, where a first capture probe of the first set of capture probes includes: (i) a first spatial barcode; (ii) a first capture domain; and (iii) a first photocleavable cleavage site; and where a second capture probe of the second set of capture probes includes: (i) a second spatial barcode; (ii) a second capture domain; and (iii) a second photocleavable cleavage site; where the first photocleavable cleavage site of the first capture probe differs from the second photocleavable cleavage site of the second capture probe.

In some embodiments, the composition includes the first capture probe released from the array and hybridized to an analyte in a biological sample. In some embodiments, the composition includes an extended first capture probe where the analyte was used as a template.

In some embodiments, the composition includes the analyte hybridized to the first capture probe on the array. In some embodiments, the composition includes an extended first capture probe on the array, where the analyte was used as a template.

In some embodiments, the composition includes a second capture probe hybridized to a second analyte or an analyte from a second biological sample. In some embodiments, the second capture probe was released prior to hybridizing to the second analyte or the analyte from the second biological sample. In some embodiments, the second capture probe was released after hybridizing to the second analyte or the analyte from the second biological sample.

In some embodiments, the composition includes an RNase, one or more permeabilization reagents, a polymerase, a reverse transcriptase, a cross-linking agent, and combinations thereof.

In some embodiments, the composition includes a plurality of first probes and a plurality of second probes, where a first probe of the plurality of first probes and a second probe of the plurality of second probes each include a sequence that is substantially complementary to sequences of the analyte, and where the second probe includes a capture probe capture domain.

In some embodiments, the first probe and the second probe are coupled to form a ligation product with the use of a ligase.

In some embodiments, the composition includes a first capture probe released from the array and hybridized to the ligation product in a biological sample. In some embodiments, the composition includes an extended first capture probe using the ligation product as a template.

In some embodiments, the composition includes a hybridized ligation product to the first capture probe on the array. In some embodiments, the composition includes an extended first capture probe using the ligation product as a template on the array.

In some embodiments, the composition includes a plurality of analyte capture agents, where an analyte capture agent includes: (i) an analyte binding moiety and (ii) an oligonucleotide including an analyte binding moiety barcode and an analyte capture sequence.

In some embodiments, the composition includes a second capture probe released from the array and hybridized to the analyte capture sequence of the analyte capture agent in a biological sample. In some embodiments, the composition includes an extended second capture probe where the analyte capture sequence was used as a template.

In some embodiments, the array includes one or more additional sets of capture probes.

Also provided herein are methods for determining the spatial location of an analyte in a biological sample, the method including: (a) contacting the biological with an array, where the array includes a first set of capture probes and a second set of capture probes, where a first capture probe of the first set of capture probes includes: (i) a first spatial barcode; (ii) a first capture domain; and (iii) a first restriction endonuclease cleavage site; and where a second capture probe of the second set of capture probes includes: (i) a second spatial barcode; (ii) a second capture domain; and (iii) a second restriction endonuclease cleavage site; where the sequence of the first restriction endonuclease cleavage site of the first capture probe differs from the sequence of the second restriction endonuclease cleavage site of the second capture probe; (b) hybridizing the analyte to the first capture domain; (c) releasing the first set of capture probes from the array; and (d) determining (i) all or a part of the sequence of the analyte, or a complement thereof, and (ii) the sequence of the first spatial barcode, or a complement thereof, and using the determined sequences of (i) and (ii) to determine the spatial location of the analyte in the biological sample.

Also provided herein are methods for determining the spatial location of an analyte in a biological sample, the method including: (a) contacting the biological with an array, where the array includes a first set of capture probes and a second set of capture probes, where a first capture probe of the first set of capture probes includes: (i) a first spatial barcode; (ii) a first capture domain; and (iii) a first photocleavable cleavage site; and where a second capture probe of the second set of capture probes includes: (i) a second spatial barcode; (ii) a second capture domain; and (iii) a second photocleavable cleavage site; where the first photocleavable cleavage site of the first capture probe differs from the second photocleavable cleavage site of the second capture probe; (b) hybridizing the analyte to the first capture domain; (c) releasing the first set of capture probes from the array; and (d) determining (i) all or a part of the sequence of the analyte, or a complement thereof, and (ii) the sequence of the first spatial barcode, or a complement thereof, and using the determined sequences of (i) and (ii) to determine the spatial location of the analyte in the biological sample.

Also provided herein are methods for determining the spatial location of an analyte in a biological sample, the method including: (a) contacting the biological sample with an array, where the array includes a first set of capture probes and a second set of capture probes, where a first capture probe of the first set of capture probes includes: (i) a first spatial barcode; (ii) a first capture domain; and (iii) a first restriction endonuclease cleavage site; and where a second capture probe of the second set of capture probes includes: (i) a second spatial barcode; (ii) a second capture domain; and (iii) a second restriction endonuclease cleavage site; where the sequence of the first restriction endonuclease cleavage site of the first capture probe differs from the sequence of the second restriction endonuclease cleavage site of the second capture probe; (b) hybridizing a first probe and a second probe to the analyte, where the first probe and the second probe each include a sequence that is substantially complementary to sequences of the analyte, and where the second probe includes a capture probe binding domain; (c) coupling the first probe and the second probe, thereby generating a ligation product; (d) hybridizing the ligation product to the first capture domain of the first capture probe; (c) releasing the first set of determining (i) all or a part of the sequence of the capture probes from the array; and (f) analyte, or a complement thereof, and (ii) the sequence of the first spatial barcode, or a complement thereof, and using the determined sequences of (i) and (ii) to determine the spatial location of the analyte in the biological sample.

Also provided herein are methods for determining the spatial location of an analyte in a biological sample, the method including: (a) contacting the biological sample with an array, where the array includes a first set of capture probes and a second set of capture probes, where a first capture probe of the first set of capture probes includes: (i) a first spatial barcode; (ii) a first capture domain; and (iii) a first photocleavable cleavage site; and where a second capture probe of the second set of capture probes includes: (i) a second spatial barcode; (ii) a second capture domain; and (iii) a second photocleavable cleavage site; where the first photocleavable site of the first capture probe differs from the second photocleavable site of the second capture probe; (b) hybridizing a first probe and a second probe to the analyte, where the first probe and the second probe each include a sequence that is substantially complementary to adjacent sequences of the analyte, and where the second probe includes a capture probe binding domain; (c) coupling the first probe and the second probe, thereby generating a ligation product; (d) hybridizing the ligation product to the first capture domain of the first capture probe; (c) releasing the first set of capture probes from the array; and (f) determining (i) all or a part of the sequence of the analyte, or a complement thereof, and (ii) the sequence of the first spatial barcode, or a complement thereof, and using the determined sequences of (i) and (ii) to determine the spatial location of the analyte in the biological sample.

In some embodiments, the method includes contacting the biological sample with one or more analyte capture agents, where an analyte capture agent includes: (i) an analyte binding moiety and (ii) an oligonucleotide including an analyte binding moiety barcode and an analyte capture sequence.

In some embodiments, the analyte binding moiety barcode identifies the analyte, optionally where the analyte is a protein.

In some embodiments, the analyte capture sequence hybridizes to the second capture domain of the second capture probe, optionally where the analyte binding moiety is an antibody or an antigen binding fragment thereof.

All publications, patents, and patent applications mentioned in this specification are herein incorporated by reference to the same extent as if each individual publication, patent, patent application, or item of information was specifically and individually indicated to be incorporated by reference. To the extent publications, patents, patent applications, and items of information incorporated by reference contradict the disclosure contained in the specification, the specification is intended to supersede and/or take precedence over any such contradictory material.

Where values are described in terms of ranges, it should be understood that the description includes the disclosure of all possible sub-ranges within such ranges, as well as specific numerical values that fall within such ranges irrespective of whether a specific numerical value or specific sub-range is expressly stated.

The term “about” or “approximately” as used herein means within an acceptable error range for the particular value as determined by one of ordinary skill in the art, which will depend in part on how the value is measured or determined, i.e., the limitations of the measurement system. For example, “about” can mean within an acceptable standard deviation, per the practice in the art. Alternatively, “about” can mean a range of up to +20%, preferably up to +10%, more preferably up to +5%, and more preferably still up to +1% of a given value. Alternatively, particularly with respect to biological systems or processes, the term can mean within an order of magnitude, preferably within 2-fold, of a value. Where particular values are described in the application and claims, unless otherwise stated, the term “about” is implicit and in this context means within an acceptable error range for the particular value.

The term “substantially complementary” used herein means that a first sequence is at least 50%, 55%, 60%, 65%, 70%, 75%, 80%, 85%, 90%, 95%, 97%, 98%, or 99% identical to the complement of a second sequence over a region of 2, 3, 4, 5, 6, 7, 8, 9, 10, 11, 12, 13, 14, 15, 16, 17, 18, 19, 20-40, 40-60, 60-100, or more nucleotides, or that the two sequences hybridize under stringent hybridization conditions. Substantially complementary also means that a sequence in one strand is not completely and/or perfectly complementary to a sequence in an opposing strand, but that sufficient bonding occurs between bases on the two strands to form a stable hybrid complex in set of hybridization conditions (e.g., salt concentration and temperature). Such conditions can be predicted by using the sequences and standard mathematical calculations known to those skilled in the art.

The term “each,” when used in reference to a collection of items, is intended to identify an individual item in the collection but does not necessarily refer to every item in the collection, unless expressly stated otherwise, or unless the context of the usage clearly indicates otherwise.

Various embodiments of the features of this disclosure are described herein. However, it should be understood that such embodiments are provided merely by way of example, and numerous variations, changes, and substitutions can occur to those skilled in the art without departing from the scope of this disclosure. It should also be understood that various alternatives to the specific embodiments described herein are also within the scope of this disclosure.

### DESCRIPTION OF DRAWINGS

The following drawings illustrate certain embodiments of the features and advantages of this disclosure. These embodiments are not intended to limit the scope of the appended claims in any manner. Like reference symbols in the drawings indicate like elements.

FIG. 1A shows an exemplary sandwiching process where a first substrate (e.g., a slide), including a biological sample, and a second substrate (e.g., array slide) are brought into proximity with one another.

FIG. 1B shows a fully formed sandwich configuration creating a chamber formed from the one or more spacers, the first substrate, and the second substrate.

FIG. 2A shows a perspective view of an exemplary sample handling apparatus in a closed position.

FIG. 2B shows a perspective view of an exemplary sample handling apparatus in an open position.

FIG. 3A shows the first substrate angled over (superior to) the second substrate.

FIG. 3B shows that as the first substrate lowers, and/or as the second substrate rises, the dropped side of the first substrate may contact a drop of reagent medium.

FIG. 3C shows a full closure of the sandwich between the first substrate and the second substrate with one or more spacers contacting both the first substrate and the second substrate.

FIG. 4A shows a side view of the angled closure workflow.

FIG. 4B shows a top view of the angled closure workflow.

FIG. 5 is a schematic diagram showing an example of a barcoded capture probe, as described herein.

FIG. 6 shows a schematic illustrating a cleavable capture probe.

FIG. 7 shows exemplary capture domains on capture probes.

FIG. 8 shows an exemplary arrangement of barcoded features within an array.

FIG. 9A shows an exemplary workflow for performing templated capture and producing a ligation product.

FIG. 9B shows an exemplary workflow for capturing a ligation product from FIG. 9A on a substrate.

FIG. 10 is a schematic diagram of an exemplary analyte capture agent.

FIG. 11 is a schematic diagram depicting an exemplary interaction between a feature-immobilized capture probe 1124 and an analyte capture agent 1126.

FIG. 12 shows an exemplary photocleavable oligonucleotide including a 5′-PC-amino modifier CE phosphoramidite.

FIG. 13 is a table providing exemplary photoreactive groups, and their structures, that can be incorporated into the disclosed capture probes.

### DETAILED DESCRIPTION

### A. Spatial Analysis Methods

Spatial analysis methodologies described herein can provide a vast amount of analyte and/or expression data for a variety of analytes within a biological sample at high spatial resolution, while retaining native spatial context. Spatial analysis methods can include, e.g., the use of a capture probe including a spatial barcode (e.g., a nucleic acid sequence that provides information as to the location or position of an analyte within a cell or a tissue sample (e.g., mammalian cell or a mammalian tissue sample) and a capture domain that is capable of binding to an analyte (e.g., a protein and/or a nucleic acid) produced by and/or present in a cell. Spatial analysis methods and compositions can also include the use of a capture probe having a capture domain that captures an intermediate agent for indirect detection of an analyte. For example, the intermediate agent can include a nucleic acid sequence (e.g., a barcode) associated with the intermediate agent. Detection of the intermediate agent is therefore indicative of the analyte in the cell or tissue sample.

Non-limiting aspects of spatial analysis methodologies and compositions are described in U.S. Pat. Nos. 11,447,807, 11,352,667, 11,168,350, 11,104,936, 11,008,608, 10,995,361, 10,913,975, 10,774,374, 10,724,078, 10,640,816, 10,494,662, 10,480,022, 10,364,457, 10,317,321, 10,059,990, 10,041,949, 10,030,261, 10,002,316, 9,879,313, 9,783,841, 9,727,810, 9,593,365, 8,951,726, 8,604,182, and 7,709,198; U.S. Patent Application Publication Nos. 2020/0239946, 2020/0080136, 2020/0277663, 2019/0330617, 2020/0256867, 2020/0224244, 2019/0085383, and 2013/0171621; PCT Publication Nos. WO2018/091676, WO2020/176788, WO2017/144338, and WO2016/057552; Non-patent literature references Rodriques et al., Science 363 (6434): 1463-1467, 2019; Lee et al., Nat. Protoc. 10 (3):442-458, 2015; Trejo et al., PLOS ONE 14 (2):e0212031, 2019; Chen et al., Science 348 (6233):aaa6090, 2015; Gao et al., BMC Biol. 15:50, 2017; and Gupta et al., Nature Biotechnol. 36:1197-1202, 2018; and the Visium Spatial Gene Expression Reagent Kits User Guide (e.g., Rev F, dated January 2022); and/or the Visium Spatial Gene Expression Reagent Kits-Tissue Optimization User Guide (e.g., Rev E, dated February 2022), both of which are available at the 10× Genomics Support Documentation website, and can be used herein in any combination, and each of which is incorporated herein by reference in its entirety. Further non-limiting aspects of spatial analysis methodologies and compositions are described herein.

Some general terminology that may be used in this disclosure can be found in Section (I) (b) of PCT Publication No. WO2020/176788 and/or U.S. Patent Application Publication No. 2020/0277663, which is herein incorporated by reference. Typically, a “barcode” is a label, or identifier, that conveys or is capable of conveying information (e.g., information about an analyte in a sample, a bead, and/or a capture probe). A barcode can be part of an analyte, or independent of an analyte. A barcode can be attached to an analyte. A particular barcode can be unique relative to other barcodes. For the purpose of this disclosure, an “analyte” can include any biological substance, structure, moiety, or component to be analyzed. The term “target” can similarly refer to an analyte of interest.

Analytes can be broadly classified into one of two groups: nucleic acid analytes and non-nucleic acid analytes. Examples of non-nucleic acid analytes include, but are not limited to, lipids, carbohydrates, peptides, proteins, glycoproteins (N-linked or O-linked), lipoproteins, phosphoproteins, specific phosphorylated or acetylated variants of proteins, amidation variants of proteins, hydroxylation variants of proteins, methylation variants of proteins, ubiquitylation variants of proteins, sulfation variants of proteins, viral proteins (e.g., viral capsid, viral envelope, viral coat, viral accessory, viral glycoproteins, viral spike, etc.), extracellular and intracellular proteins, antibodies, and antigen binding fragments. In some embodiments, the analyte(s) can be localized to subcellular location(s), including, for example, organelles, e.g., mitochondria, Golgi apparatus, endoplasmic reticulum, chloroplasts, endocytic vesicles, exocytic vesicles, vacuoles, lysosomes, etc. In some embodiments, analyte(s) can be peptides or proteins, including without limitation antibodies and enzymes. Additional examples of analytes can be found in Section (I) (c) of PCT Publication No. WO2020/176788 and/or U.S. Patent Application Publication No. 2020/0277663, which is herein incorporated by reference. In some embodiments, an analyte can be detected indirectly, such as through detection of an intermediate agent, for example, a ligation product or an analyte capture agent (e.g., an oligonucleotide-conjugated antibody), such as those described herein.

A “biological sample” is typically obtained from the subject for analysis using any of a variety of techniques including, but not limited to, biopsy, surgery, and laser capture microscopy (LCM), and generally includes cells and/or other biological material from the subject. In some embodiments, the biological sample is a tissue sample. In some embodiments, the biological sample (e.g., tissue sample) is a tissue microarray (TMA). A tissue microarray contains multiple representative tissue samples-which can be from different tissues or organisms-assembled on a single histologic slide. The TMA can therefore allow for high throughput analysis of multiple specimens at the same time. Tissue microarrays are paraffin blocks produced by extracting cylindrical tissue cores from different paraffin donor blocks and re-embedding these tissue cores into a single recipient (microarray) block at defined array coordinates.

The biological sample as used herein can be any suitable biological sample described herein or known in the art. In some embodiments, the biological sample is a tissue sample. In some embodiments, the tissue sample is a solid tissue sample. In some embodiments, the biological sample is a tissue section. In some embodiments, the tissue is flash-frozen and sectioned. Any suitable method described herein or known in the art can be used to flash-freeze and section the tissue sample. In some embodiments, the biological sample, e.g., the tissue, is flash-frozen using liquid nitrogen before sectioning. In some embodiments, the biological sample, e.g., a tissue sample, is flash-frozen using nitrogen (e.g., liquid nitrogen), isopentane, or hexane.

In some embodiments, the biological sample, e.g., the tissue, is embedded in a matrix e.g., optimal cutting temperature (OCT) compound to facilitate sectioning. OCT compound is a formulation of clear, water-soluble glycols and resins, providing a solid matrix to encapsulate biological (e.g., tissue) specimens. In some embodiments, the sectioning is performed by cryosectioning, for example using a microtome. In some embodiments, the methods further comprise a thawing step, after the cryosectioning.

The biological sample can be from a mammal. In some instances, the biological sample is from a human, mouse, or rat. In addition to the subjects described above, the biological sample can be obtained from non-mammalian organisms (e.g., a plant, an insect, an arachnid, a nematode (e.g., Caenorhabditis elegans), a fungus, an amphibian, or a fish (e.g., zebrafish)). A biological sample can be obtained from a prokaryote such as a bacterium, e.g., Escherichia coli, Staphylococci or Mycoplasma pneumoniae; an archacon; a virus, such as Hepatitis C virus or human immunodeficiency virus; or a viroid. A biological sample can be obtained from a eukaryote, such as a patient derived organoid (PDO) or patient derived xenograft (PDX). The biological sample can include organoids, a miniaturized and simplified version of an organ produced in vitro in three dimensions that shows realistic micro-anatomy. Organoids can be generated from one or more cells from a tissue, embryonic stem cells, and/or induced pluripotent stem cells, which can self-organize in three-dimensional culture owing to their self-renewal and differentiation capacities. In some embodiments, an organoid is a cerebral organoid, an intestinal organoid, a stomach organoid, a lingual organoid, a thyroid organoid, a thymic organoid, a testicular organoid, a hepatic organoid, a pancreatic organoid, an epithelial organoid, a lung organoid, a kidney organoid, a gastruloid, a cardiac organoid, or a retinal organoid. Subjects from which biological samples can be obtained can be healthy or asymptomatic individuals, individuals that have or are suspected of having a disease (e.g., cancer) or a pre-disposition to a disease, and/or individuals that are in need of therapy or suspected of needing therapy.

Biological samples can be derived from a homogeneous culture or population of the subjects or organisms mentioned herein or alternatively from a collection of several different organisms, for example, in a community or ecosystem.

Biological samples can include one or more diseased cells. A diseased cell can have altered metabolic properties, gene expression, protein expression, and/or morphologic features. Examples of diseases include inflammatory disorders, metabolic disorders, nervous system disorders, and cancer. Cancer cells can be derived from solid tumors, hematological malignancies, cell lines, or obtained as circulating tumor cells.

In some embodiments, the biological sample, e.g., the tissue sample, is fixed in a fixative including alcohol, for example methanol. In some embodiments, instead of methanol, acetone, or an acetone-methanol mixture can be used. In some embodiments, the fixation is performed after sectioning. In some instances, when the biological sample is fixed using a fixative including an alcohol (e.g., methanol or acetone-methanol mixture), the biological sample is not decrosslinked afterward. In some preferred embodiments, the biological sample is fixed using a fixative including an alcohol (e.g., methanol or an acetone-methanol mixture) after freezing and/or sectioning. In some instances, the biological sample is flash-frozen, and then the biological sample is sectioned and fixed (e.g., using methanol, acetone, or an acetone-methanol mixture). In some instances when methanol, acetone, or an acetone-methanol mixture is used to fix the biological sample, the sample is not decrosslinked at a later step. In instances when the biological sample is frozen (e.g., flash frozen using liquid nitrogen and embedded in OCT) followed by sectioning and alcohol (e.g., methanol, acetone-methanol) fixation or acetone fixation, the biological sample is referred to as “fresh frozen”. In some embodiments, fixation of the biological sample e.g., using acetone and/or alcohol (e.g., methanol, acetone-methanol) is performed while the sample is mounted on a substrate (e.g., glass slide, such as a positively charged glass slide).

In some embodiments, the biological sample, e.g., the tissue sample, is fixed e.g., immediately after being harvested from a subject. In such embodiments, the fixative is preferably an aldehyde fixative, such as paraformaldehyde (PFA) or formalin. In some embodiments, the fixative induces crosslinks within the biological sample. In some embodiments, after fixing e.g., by formalin or PFA, the biological sample is dehydrated via sucrose gradient. In some instances, the fixed biological sample is treated with a sucrose gradient and then embedded in a matrix e.g., OCT compound. In some instances, the fixed biological sample is not treated with a sucrose gradient, but rather is embedded in a matrix e.g., OCT compound after fixation. In some embodiments, when a fixed frozen tissue sample is treated with a sucrose gradient, the sample can be rehydrated with an ethanol gradient. In some embodiments, the PFA or formalin fixed biological sample, which can be optionally dehydrated via sucrose gradient and/or embedded in OCT compound, is then frozen e.g., for storage or shipment. In such instances, the biological sample is referred to as “fixed frozen”. In preferred embodiments, a fixed frozen biological sample is not treated with methanol. In preferred embodiments, a fixed frozen biological sample is not paraffin embedded. Thus, in preferred embodiments, a fixed frozen biological sample is not deparaffinized. In some embodiments, a fixed frozen biological sample is rehydrated using an ethanol gradient.

In some instances, the biological sample (e.g., a fixed frozen tissue sample) is treated with a citrate buffer. Citrate buffer can be used to decrosslink antigens and fixation medium in the biological sample for antigen retrieval. Thus, any suitable decrosslinking agent can be used in addition to or alternatively to citrate buffer. In some embodiments, for example, the biological sample (e.g., a fixed frozen tissue sample) is decrosslinked using TE buffer.

In any of the foregoing, the biological sample can further be stained, imaged, and/or destained. For example, in some embodiments, a fresh frozen tissue sample or fixed frozen tissue sample is stained (e.g., via cosin and/or hematoxylin), imaged, destained (e.g., via HCl), or a combination thereof. In some embodiments, when a fresh frozen tissue sample is fixed in methanol, the sample is treated with isopropanol prior to being stained (e.g., via cosin and/or hematoxylin), imaged, destained (e.g., via HCl), or a combination thereof. In some embodiments when a fixed frozen tissue sample is treated with a sucrose gradient, the sample can be rehydrated using an ethanol gradient before being stained, (e.g., via cosin and/or hematoxylin), imaged, destained (e.g., via HCl), decrosslinked (e.g., via TE buffer or citrate buffer), or a combination thereof. In some embodiments, the biological sample can undergo further fixation (e.g., while mounted on a substrate), stained, imaged, and/or destained. For example, a fixed frozen biological sample may be subject to an additional fixing step (e.g., using PFA) before optional ethanol rehydration, staining, imaging, and/or destaining.

In any of the foregoing, the biological sample can be fixed using PAXgene. For example, the biological sample can be fixed using PAXgene in addition, or alternatively to, a fixative disclosed herein (e.g., alcohol, acetone, acetone-alcohol, formalin, paraformaldehyde). PAXgene is a non-cross-linking mixture of different alcohols, an acid, and a soluble organic compound that preserves morphology of biomolecules. PAXgene provides a two-reagent fixative system in which tissue is firstly fixed in a solution containing methanol and acetic acid then stabilized in a solution containing ethanol. See, Ergin B. et al., J Proteome Res. 2010 Oct. 1; 9 (10): 5188-96; Kap M. et al., PLOS One.; 6 (11): e27704 (2011); and Mathieson W. et al., Am J Clin Pathol.; 146 (1): 25-40 (2016), each of which is hereby incorporated by reference in its entirety, for a description and evaluation of PAXgene for tissue fixation. Thus, in some embodiments, when the biological sample, e.g., the tissue sample, is fixed in a fixative including alcohol, the fixative is PAXgene. In some embodiments, a fresh frozen tissue sample is fixed with PAXgene. In some embodiments, a fixed frozen tissue sample is fixed with PAXgene.

In some embodiments, the biological sample, e.g., the tissue sample is fixed, for example in methanol, acetone, acetone-methanol, PFA, PAXgene or is formalin-fixed and paraffin-embedded (FFPE). In some embodiments, the biological sample comprises intact cells. In some embodiments, the biological sample is a cell pellet, e.g., a fixed cell pellet, e.g., an FFPE cell pellet. FFPE samples are used in some instances in the RNA-templated ligation (RTL) methods disclosed herein. A limitation of direct RNA capture for fixed samples is that the RNA integrity of fixed (e.g., FFPE) samples can be lower than a fresh sample. As such, capturing RNA directly from fixed samples, e.g., by capture of a common sequence, such as a poly (A) tail of an mRNA molecule, can be more difficult. By utilizing RTL probes that hybridize to RNA target sequences in the transcriptome, RNA analytes can be captured without requiring that both a poly (A) tail and target sequences remain intact. Accordingly, RTL probes can be utilized to beneficially improve capture and spatial analysis of fixed samples. The biological sample, e.g., tissue sample, can be stained, and imaged prior, during, and/or after each step of the methods described herein. Any of the methods described herein or known in the art can be used to stain and/or image the biological sample. In some embodiments, the imaging occurs prior to destaining the sample. In some embodiments, the biological sample is stained using an H&E staining method. In some embodiments, the tissue sample is stained and imaged for about 10 minutes to about 2 hours (or any of the subranges of this range described herein). Additional time may be needed for staining and imaging of different types of biological samples.

The tissue sample can be obtained from any suitable location in a tissue or organ of a subject, e.g., a human subject. In some instances, the sample is a mouse sample. In some instances, the sample is a human sample. In some embodiments, the sample can be derived from skin, brain, breast, lung, liver, kidney, prostate, tonsil, thymus, testes, bone, lymph node, ovary, eye, heart, or spleen. In some instances, the sample is a human or mouse breast tissue sample. In some instances, the sample is a human or mouse brain tissue sample. In some instances, the sample is a human or mouse lung tissue sample. In some instances, the sample is a human or mouse tonsil tissue sample. In some instances, the sample is a human or mouse liver tissue sample. In some instances, the sample is a human or mouse bone, skin, kidney, thymus, testes, or prostate tissue sample. In some embodiments, the tissue sample is derived from normal or diseased tissue. In some embodiments, the sample is an embryo sample. The embryo sample can be a non-human embryo sample. In some instances, the sample is a mouse embryo sample.

Non-limiting examples of stains include histological stains (e.g., hematoxylin and/or cosin) and immunological stains (e.g., fluorescent stains). The biological sample can be stained using Can-Grunwald, Giemsa, hematoxylin and eosin (H&E), Jenner's, Leishman, Masson's trichrome, Papanicolaou, Romanowsky, silver, Sudan, Wright's, and/or Periodic Acid Schiff (PAS) staining techniques. In some instances, PAS staining is performed after formalin or acetone fixation. In some embodiments, a biological sample (e.g., a fixed and/or stained biological sample) can be imaged. Biological samples are also described in Section (I) (d) of PCT Publication No. WO2020/176788 and/or U.S. Patent Application Publication No. 2020/0277663, which is herein incorporated by reference.

The following embodiments can be used with any of the methods described herein. In some embodiments, the biological sample is imaged. In some embodiments, the biological sample is visualized or imaged using bright field microscopy. In some embodiments, the biological sample is visualized or imaged using fluorescence microscopy. The biological sample can be visualized or imaged using additional methods of visualization and imaging are known in the art. Non-limiting examples of visualization and imaging include expansion microscopy, bright field microscopy, dark field microscopy, phase contrast microscopy, electron microscopy, fluorescence microscopy, reflection microscopy, interference microscopy and confocal microscopy. In some embodiments, the sample is stained and imaged prior to adding reagents for analyzing captured analytes as disclosed herein to the biological sample.

In some embodiments, the method includes staining the biological sample. In some embodiments, the staining includes the use of hematoxylin and/or cosin. In some embodiments, a biological sample can be stained using any number of biological stains including, but not limited to, acridine orange, Bismarck brown, carmine, coomassie blue, cresyl violet, DAPI (4′,6-diamidino-2-phenylindole), cosin, ethidium bromide, acid fuchsine, hematoxylin, Hoechst stains, iodine, methyl green, methylene blue, neutral red, Nile blue, Nile red, osmium tetroxide, propidium iodide, rhodamine, or safranin. In some instances, the biological sample can be stained using known staining techniques, including Can-Grunwald, Giemsa, hematoxylin and eosin (H&E), Jenner's, Leishman, Masson's trichrome, Papanicolaou, Romanowsky, silver, Sudan, Wright's, and/or Periodic Acid Schiff (PAS) staining techniques. PAS staining is typically performed after formalin or acetone fixation.

In some embodiments, the staining includes the use of a detectable label, such as a radioisotope, a fluorophore, a chemiluminescent compound, a bioluminescent compound, or a combination thereof.

In some embodiments, a biological sample is permeabilized with one or more permeabilization reagents. For example, permeabilization of a biological sample can facilitate analyte capture. Exemplary permeabilization agents and conditions are described in Section (I)(d)(ii)(13) or the Exemplary Embodiments Section of PCT Publication No. WO2020/176788 and/or U.S. Patent Application Publication No. 2020/0277663, which is herein incorporated by reference. Briefly, in any of the methods described herein, the method includes a step of permeabilizing the biological sample. For example, the biological sample can be permeabilized to facilitate transfer of the extension products to the capture probes on the array. In some embodiments, the permeabilizing includes the use of an organic solvent (e.g., acetone, ethanol, or methanol), a detergent (e.g., saponin, Triton X-100™, Tween-20™, or sodium dodecyl sulfate (SDS)), an enzyme (e.g., an endopeptidase, an exopeptidase, or a protease), or a combination thereof. In some embodiments, the permeabilizing includes the use of an endopeptidase, a protease, SDS, polyethylene glycol tert-octylphenyl ether, polysorbate 80, and polysorbate 20, N-lauroylsarcosine sodium salt solution, saponin, Triton X-100™, Tween-20™, or a combination thereof. In some embodiments, the endopeptidase is pepsin. In some embodiments, the endopeptidase is Proteinase K. Additional methods for sample permeabilization are described, for example, in Jamur et al., Method Mol. Biol. 588:63-66, 2010, which is incorporated herein by reference.

Array-based spatial analysis methods involve the transfer of one or more analytes from a biological sample to an array of features on a substrate, where each feature is associated with a unique spatial location on the array. Subsequent analysis of the transferred analytes includes determining the identity of the analytes and the spatial location of the analytes within the biological sample. The spatial location of an analyte within the biological sample is determined based on the feature to which the analyte is bound (e.g., directly or indirectly) on the array, and the feature's relative spatial location within the array.

A “capture probe” refers to any molecule capable of capturing (directly or indirectly) and/or labelling an analyte (e.g., an analyte of interest) in a biological sample. In some embodiments, the capture probe is a nucleic acid or a polypeptide. In some embodiments, the capture probe includes a barcode (e.g., a spatial barcode and/or a unique molecular identifier (UMI) and a capture domain). In some instances, the capture probe includes a homopolymer sequence, such as a poly (T) sequence. In some embodiments, a capture probe can include a cleavage domain and/or a functional domain (e.g., a primer-binding site, such as for next-generation sequencing (NGS)). See, e.g., Section (II) (b) (e.g., subsections (i)-(vi)) of PCT Publication No. WO2020/176788 and/or U.S. Patent Application Publication No. 2020/0277663, which is herein incorporated by reference. Generation of capture probes can be achieved by any appropriate method, including those described in Section (II) (d) (ii) of PCT Publication No. WO2020/176788 and/or U.S. Patent Application Publication No. 2020/0277663, which is herein incorporated by reference.

In some instances, a capture probe and a nucleic acid analyte interaction (or any other nucleic acid to nucleic acid interaction) occurs because the sequences of the two nucleic acids are substantially complementary to one another. By “substantial,” “substantially” and the like, two nucleic acid sequences can be complementary when at least 60% of the nucleotide residues of one nucleic acid sequence are complementary to nucleotide residues of the other nucleic acid sequence. The complementary residues within a particular complementary nucleic acid sequence need not always be contiguous with each other, but can be interrupted by one or more non-complementary residues within the complementary nucleic acid sequence. In some embodiments, at least 60%, but less than 100%, of the residues of one of the two complementary nucleic acid sequences are complementary to residues of the other nucleic acid sequence. In some embodiments, at least 70%, 80%, 90%, 95%, or 99% of the residues of one nucleic acid sequence are complementary to residues of the other nucleic acid sequence. Sequences are said to be “substantially complementary” when at least 60% (e.g., at least 70%, at least 80%, or at least 90%) of the residues of one nucleic acid sequence are complementary to residues in the other nucleic acid sequence.

In some embodiments, the biological sample is mounted on a first substrate and the substrate comprising the array of capture probes is a second substrate. In this configuration, one or more analytes or analyte derivatives (e.g., intermediate agents, e.g., ligation products) can then be released from the biological sample and migrate to the second substrate comprising an array of capture probes. In some embodiments, the release and migration of the analytes or analyte derivatives to the second substrate comprising the array of capture probes occurs in a manner that preserves the original spatial context of the analytes in the biological sample. This method can be referred to as a sandwiching process, which is described e.g., in U.S. Patent Application Publication No. 2021/0189475 and PCT Publication Nos. WO 2021/252747 A1, WO 2022/061152 A2, and WO 2022/140028 A1, each of which is herein incorporated by reference.

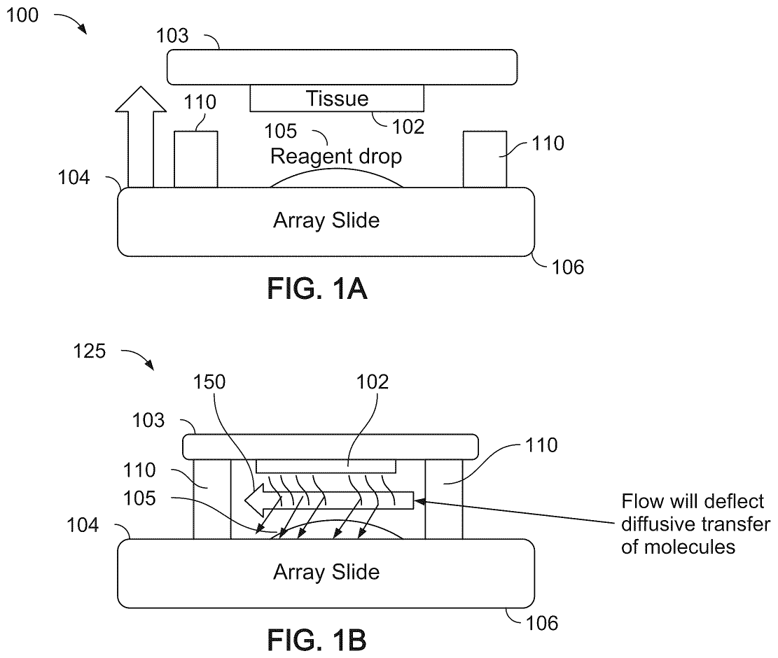

FIG. 1A shows an exemplary sandwiching process 100 where a first substrate (e.g., slide 103), including a biological sample 102, and a second substrate (e.g., array slide 104 including an array having spatially barcoded capture probes 106) are brought into proximity with one another. As shown in FIG. 1A, a liquid reagent drop (e.g., permeabilization solution 105) is introduced on the second substrate in proximity to the capture probes 106 and in between the biological sample 102 and the second substrate (e.g., slide 104 including an array having spatially barcoded capture probes 106). The permeabilization solution 105 may release analytes or analyte derivatives (e.g., intermediate agents, e.g., ligation products) that can be captured by the capture probes of the array 106.

During the exemplary sandwiching process, the first substrate is aligned with the second substrate, such that at least a portion of the biological sample is aligned with at least a portion of the capture probes (e.g., aligned in a sandwich configuration). As shown, the second substrate (e.g., array slide 104) is in an inferior position to the first substrate (e.g., slide 103). In some embodiments, the first substrate (e.g., slide 103) may be positioned superior to the second substrate (e.g., slide 104). A reagent medium 105 within a gap between the first substrate (e.g., slide 103) and the second substrate (e.g., slide 104) creates a liquid interface between the two substrates. The reagent medium may be a permeabilization solution which permeabilizes and/or digests the biological sample 102. In some embodiments, wherein the biological sample 102 has been pre-permeabilized, the reagent medium is not a permeabilization solution. In some embodiments, analytes (e.g., mRNA transcripts) and/or analyte derivatives (e.g., intermediate agents, e.g., ligation products) of the biological sample 102 may release from the biological sample, and actively or passively migrate (e.g., diffuse) across the gap toward the capture probes on the array 106. Alternatively, in certain embodiments, migration of the analyte or analyte derivative (e.g., intermediate agent; e.g., ligation product) from the biological sample is performed actively (e.g., electrophoretic, by applying an electric field to promote migration). Exemplary methods of electrophoretic migration are described in WO 2020/176788, and U.S. Patent Application Publication No. 2021/0189475, each of which is hereby incorporated by reference.

As further shown, one or more spacers 110 may be positioned between the first substrate (e.g., slide 103) and the second substrate (e.g., array slide 104 including spatially barcoded capture probes 106). The one or more spacers 110 may be configured to maintain a separation distance between the first substrate and the second substrate. While the one or more spacers 110 is shown as disposed on the second substrate, the spacer may additionally or alternatively be disposed on the first substrate.

In some embodiments, the one or more spacers 110 is configured to maintain a separation distance between first and second substrates that is between about 2 microns and 1 mm (e.g., between about 2 microns and 800 microns, between about 2 microns and 700 microns, between about 2 microns and 600 microns, between about 2 microns and 500 microns, between about 2 microns and 400 microns, between about 2 microns and 300 microns, between about 2 microns and 200 microns, between about 2 microns and 100 microns, between about 2 microns and 25 microns, or between about 2 microns and 10 microns), measured in a direction orthogonal to the surface of first substrate that supports the biological sample. In some instances, the separation distance is about 2, 3, 4, 5, 6, 7, 8, 9, 10, 11, 12, 13, 14, 15, 16, 17, 18, 19, 20, 21, 22, 23, 24, or 25 microns. In some embodiments, the separation distance is less than 50 microns. In some embodiments, the separation distance is less than 25 microns. In some embodiments, the separation distance is less than 20 microns. The separation distance may include a distance of at least 2 μm.

FIG. 1B shows a fully formed sandwich configuration 125 creating a chamber 150 formed from the one or more spacers 110, the first substrate (e.g., the slide 103), and the second substrate (e.g., the slide 104 including an array 106 having spatially barcoded capture probes) in accordance with some example implementations. In the example of FIG. 1B, the liquid reagent (e.g., the permeabilization solution 105) fills the volume of the chamber 150 and may create a permeabilization buffer that allows analytes (e.g., mRNA transcripts and/or other molecules) or analyte derivatives (e.g., intermediate agents, e.g., ligation products) to diffuse from the biological sample 102 toward the capture probes of the second substrate (e.g., slide 104). In some aspects, flow of the permeabilization buffer may deflect transcripts and/or molecules from the biological sample 102 and may affect diffusive transfer of analytes or analyte derivatives (e.g., intermediate agents, e.g., ligation products) for spatial analysis. A partially or fully scaled chamber 150 resulting from the one or more spacers 110, the first substrate (e.g., slide 103), and the second substrate (e.g., slide 104) may reduce or prevent flow from undesirable movement (e.g., convective movement) of transcripts and/or molecules during the diffusive transfer from the biological sample 102 to the capture probes.

The sandwiching process methods described above can be implemented using a variety of hardware components. For example, the sandwiching process methods can be implemented using a sample holder (also referred to herein as a support device, a sample handling apparatus, and an array alignment device). Further details on support devices, sample holders, sample handling apparatuses, or systems for implementing a sandwiching process are described in, e.g., US. Patent Application Publication No. 2021/0189475, and PCT Publication No. WO 2022/061152 A2, each of which is incorporated by reference in its entirety.

In some embodiments of a sample holder, the sample holder can include a first member including a first retaining mechanism configured to retain a first substrate comprising a biological sample. The first retaining mechanism can be configured to retain the first substrate disposed in a first plane. The sample holder can further include a second member including a second retaining mechanism configured to retain a second substrate disposed in a second plane. The sample holder can further include an alignment mechanism connected to one or both of the first member and the second member. The alignment mechanism can be configured to align the first and second members along the first plane and/or the second plane such that the sample contacts at least a portion of the reagent medium when the first and second members are aligned and within a threshold distance along an axis orthogonal to the second plane. The adjustment mechanism may be configured to move the second member along the axis orthogonal to the second plane and/or move the first member along an axis orthogonal to the first plane.

In some embodiments, the adjustment mechanism includes a linear actuator. In some embodiments, the linear actuator is configured to move the second member along an axis orthogonal to the plane of the first member and/or the second member. In some embodiments, the linear actuator is configured to move the first member along an axis orthogonal to the plane of the first member and/or the second member. In some embodiments, the linear actuator is configured to move the first member, the second member, or both the first member and the second member at a velocity of at least 0.1 mm/sec. In some embodiments, the linear actuator is configured to move the first member, the second member, or both the first member and the second member with an amount of force of at least 0.1 lbs.

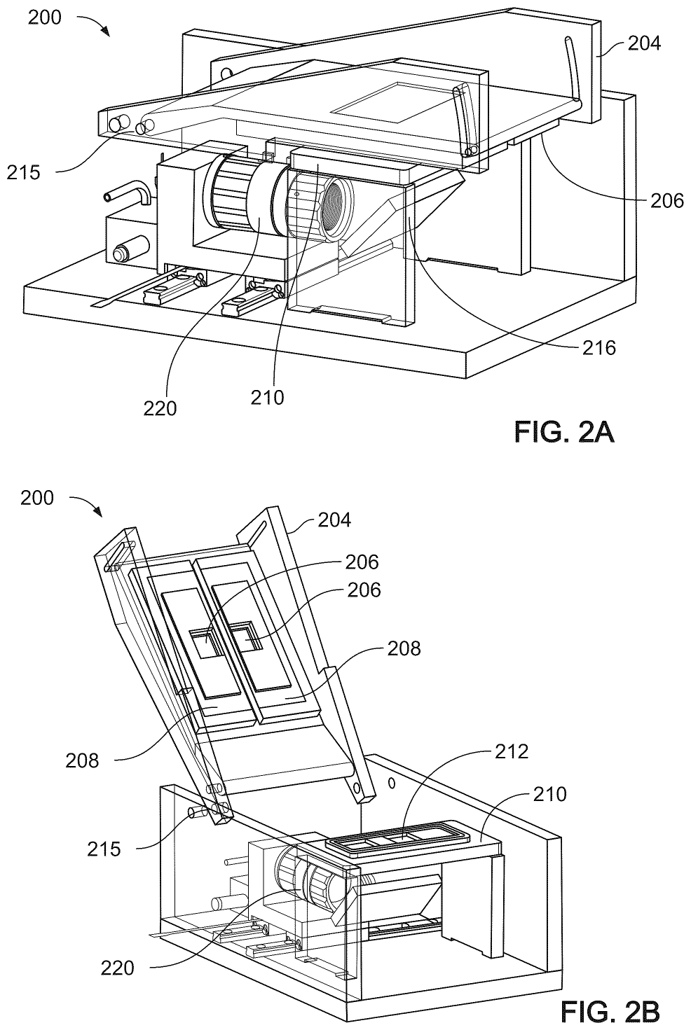

FIG. 2A is a perspective view of an example sample handling apparatus 200 in a closed position in accordance with some example implementations. As shown, the sample handling apparatus 200 includes a first member 204, a second member 210, optionally an image capture device 220, a first substrate 206, optionally a hinge 215, and optionally a mirror 216. The hinge 215 may be configured to allow the first member 204 to be positioned in an open or closed configuration by opening and/or closing the first member 204 in a clamshell manner along the hinge 215.

FIG. 2B is a perspective view of the example sample handling apparatus 200 in an open position in accordance with some example implementations. As shown, the sample handling apparatus 200 includes one or more first retaining mechanisms 208 configured to retain one or more first substrates 206. In the example of FIG. 2B, the first member 204 is configured to retain two first substrates 206, however the first member 204 may be configured to retain more or fewer first substrates 206.

In some aspects, when the sample handling apparatus 200 is in an open position (e.g., in FIG. 2B), the first substrate 206 and/or the second substrate 212 may be loaded and positioned within the sample handling apparatus 200, such as within the first member 204 and the second member 210, respectively. As noted, the hinge 215 may allow the first member 204 to close over the second member 210 and form a sandwich configuration.

In some aspects, after the first member 204 closes over the second member 210, an adjustment mechanism of the sample handling apparatus 200 may actuate the first member 204 and/or the second member 210 to form the sandwich configuration for the permeabilization of the sample (e.g., bringing the first substrate 206 and the second substrate 212 closer to each other and within a threshold distance for the sandwich configuration). The adjustment mechanism may be configured to control a speed, an angle, a force, or the like of the sandwich configuration.

In some embodiments, the biological sample (e.g., sample 102 from FIG. 1A) may be aligned within the first member 204 (e.g., via the first retaining mechanism 208) prior to closing the first member 204 such that a desired region of interest of the sample is aligned with the barcoded array of the second substrate (e.g., the slide 104 from FIG. 1A), e.g., when the first and second substrates are aligned in the sandwich configuration. Such alignment may be accomplished manually (e.g., by a user) or automatically (e.g., via an automated alignment mechanism). After or before alignment, spacers may be applied to the first substrate 206 and/or the second substrate 212 to maintain a minimum spacing between the first substrate 206 and the second substrate 212 during sandwiching. In some aspects, the permeabilization solution (e.g., permeabilization solution 305) may be applied to the first substrate 206 and/or the second substrate 212. The first member 204 may then close over the second member 210 and form the sandwich configuration. Analytes or analyte derivatives (e.g., intermediate agents, e.g., ligation products) may be captured by the capture probes of the array and may be processed for spatial analysis.

In some embodiments, during permeabilization, the image capture device 220 may capture images of the overlap area between the biological sample and the capture probes on the array 106. If more than one first substrates 206 and/or second substrates 212 are present within the sample handling apparatus 200, the image capture device 220 may be configured to capture one or more images of one or more overlap areas.

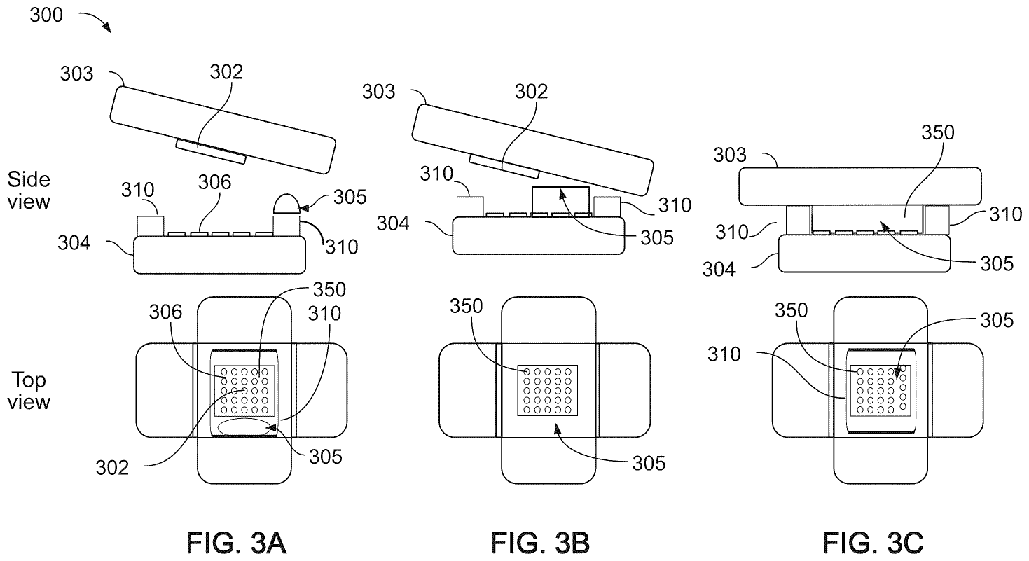

Provided herein are methods for delivering a fluid to a biological sample disposed on an area of a first substrate and an array disposed on a second substrate. FIGS. 3A-3C depict a side view and a top view of an exemplary angled closure workflow 300 for sandwiching a first substrate (e.g., slide 303) having a biological sample 302 and a second substrate (e.g., slide 304 having capture probes 306) in accordance with some exemplary implementations.

FIG. 3A depicts the first substrate (e.g., slide 303 including a biological sample 302) angled over (superior to) the second substrate (e.g., slide 304). As shown, reagent medium (e.g., permeabilization solution) 305 is located on the spacer 310 toward the right-hand side of the side view in FIG. 3A. While FIG. 3A depicts the reagent medium on the right-hand side of side view, it should be understood that such depiction is not meant to be limiting as to the location of the reagent medium on the spacer.

FIG. 3B shows that as the first substrate lowers, and/or as the second substrate rises, the dropped side of the first substrate (e.g., a side of the slide 303 angled toward the slide 304) may contact the reagent medium 305. The dropped side of the slide 303 may urge the reagent medium 305 toward the opposite direction (e.g., towards an opposite side of the spacer 310, towards an opposite side of the slide 303 relative to the dropped side). For example, in the side view of FIG. 3B the reagent medium 305 may be urged from right to left as the sandwich is formed.

In some embodiments, the first substrate and/or the second substrate are further moved to achieve an approximately parallel arrangement of the first substrate and the second substrate.

FIG. 3C depicts a full closure of the sandwich between the first substrate and the second substrate with the spacer 310 contacting both the first substrate and the second substrate and maintaining a separation distance and optionally the approximately parallel arrangement between the two substrates. As shown in the top view of FIG. 3C, the spacer 310 fully encloses and surrounds the biological sample 302 and the capture probes 306, and the spacer 310 form the sides of chamber 350 which holds a volume of the reagent medium 305.

While FIG. 3C depicts the first substrate (e.g., the slide 303 including biological sample 302) angled over (superior to) the second substrate (e.g., slide 304) and the second substrate comprising the spacer 310, it should be understood that an exemplary angled closure workflow can include the second substrate angled over (superior to) the first substrate and the first substrate comprising the spacer 310.

It may be desirable that the reagent medium be free from air bubbles between the substrates to facilitate transfer of target analytes with spatial information. Additionally, air bubbles present between the substrates may obscure at least a portion of an image capture of a desired region of interest. Accordingly, it may be desirable to ensure or encourage suppression and/or elimination of air bubbles between the two substrates (e.g., slide 303 and slide 304) during a permeabilization step (e.g., step 104). In some aspects, bubble formation between the substrates may be reduced or eliminated using a variety of filling methods and/or closing methods. In some instances, the first substrate and the second substrate are arranged in an angled sandwich assembly as described herein. For example, during the sandwiching of the two substrates (e.g., the slide 303 and the slide 304), an angled closure workflow may be used to suppress or eliminate bubble formation.

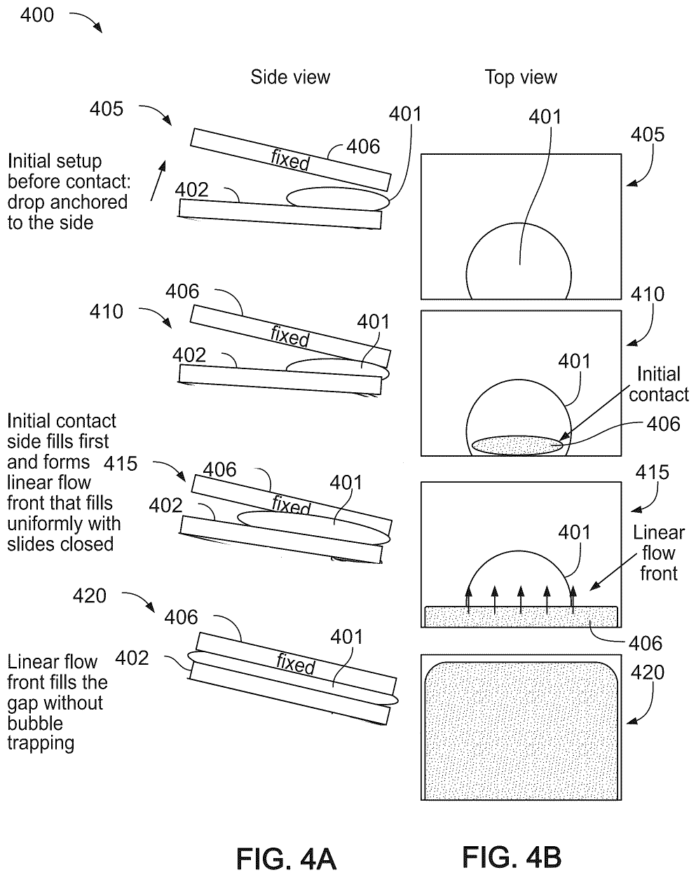

FIG. 4A is a side view of the angled closure workflow 400 in accordance with some exemplary implementations. FIG. 4B is a top view of the angled closure workflow 400 in accordance with some exemplary implementations. As shown at step 405, reagent medium 401 is positioned to the side of the substrate 402.

At step 410, the dropped side of the angled substrate 406 contacts the reagent medium 401 first. The contact of the substrate 406 with the reagent medium 401 may form a linear or low curvature flow front that fills the gap between the two substrates 406 and 402 uniformly with the slides closed.

At step 415, the substrate 406 is further lowered toward the substrate 402 (or the substrate 402 is raised up toward the substrate 406) and the dropped side of the substrate 406 may contact and may urge the reagent medium toward the side opposite the dropped side, thereby creating a linear or low curvature flow front that may prevent or reduce bubble trapping between the substrates.

At step 420, the reagent medium 401 fills the gap between the substrate 406 and the substrate 402. The linear flow front of the liquid reagent may be formed by squeezing the reagent medium 401 volume along the contact side of the substrate 402 and/or the substrate 406. Additionally, capillary flow may also contribute to filling the gap area.

In some embodiments, the reagent medium (e.g., 105 in FIG. 1A) comprises a permeabilization agent. In some embodiments, following initial contact between the biological sample and a permeabilization agent, the permeabilization agent can be removed from contact with the biological sample (e.g., by opening the sample holder). Suitable agents for this purpose include, but are not limited to, organic solvents (e.g., acetone, ethanol, or methanol), cross-linking agents (e.g., paraformaldehyde), detergents (e.g., saponin, Triton X-100™, Tween-20™, or sodium dodecyl sulfate (SDS)), and enzymes (e.g., trypsin or other proteases (e.g., proteinase K). In some embodiments, the detergent is an anionic detergent (e.g., SDS or N-lauroylsarcosine sodium salt solution).

In some embodiments, the reagent medium comprises a lysis reagent. Lysis solutions can include ionic surfactants such as, for example, sarkosyl and sodium dodecyl sulfate (SDS). More generally, chemical lysis agents can include, without limitation, organic solvents, chelating agents, detergents, surfactants, and chaotropic agents. In some embodiments, the reagent medium comprises a protease. Exemplary proteases include, e.g., pepsin, trypsin, elastase, and proteinase K. In some embodiments, the reagent medium comprises a nuclease. In some embodiments, the nuclease comprises an RNase. In some embodiments, the RNase includes RNase A, RNase C, RNase H, or RNase I. In some embodiments, the reagent medium comprises sodium dodecyl sulfate (SDS) or a sodium salt thereof, proteinase K, pepsin, N-lauroylsarcosine, or RNase.

In some embodiments, the reagent medium comprises polyethylene glycol (PEG). In some embodiments, the PEG molecular weight is from about 2K to about 16K. In some embodiments, the PEG molecular weight is about 2K, about 3K, about 4K, about 5K, about 6K, about 7K, about 8K, about 9K, about 10K, about 11K, about 12K, about 13K, about 14K, about 15K, or about 16K. In some embodiments, the PEG is present at a concentration from about 2% to about 25%, from about 4% to about 23%, from about 6% to about 21%, or from about 8% to about 20% (v/v).

In certain embodiments, a dried permeabilization reagent is applied or formed as a layer on the first substrate, the second substrate, or both prior to contacting the biological sample with the array. For example, a permeabilization reagent can be deposited in solution on the first substrate or the second substrate or both and then dried.

In some instances, the aligned portions of the biological sample and the array are in contact with the reagent medium for about 1 minute, about 5 minutes, about 10 minutes, about 12 minutes, about 15 minutes, about 18 minutes, about 20 minutes, about 25 minutes, about 30 minutes, about 36 minutes, about 45 minutes, or about an hour. In some instances, the aligned portions of the biological sample and the array are in contact with the reagent medium for about 1-60 minutes.

In some instances, the device is configured to control a temperature of the first and second substrates. In some embodiments, the temperature of the first and second members is lowered to a first temperature that is below room temperature.

There are at least two methods to associate a spatial barcode with one or more neighboring cells, such that the spatial barcode identifies the one or more cells, and/or contents of the one or more cells, as associated with a particular spatial location in a biological sample. One method is to promote analytes or analyte proxies (e.g., intermediate agents) out of a cell and towards a spatially-barcoded array (e.g., including spatially-barcoded capture probes). Another method is to release or cleave spatially-barcoded capture probes from an array and promote the spatially-barcoded capture probes towards and/or into or onto the biological sample.

In some cases, capture probes may be configured to prime, replicate, and consequently yield optionally barcoded extension products from a template (e.g., a DNA or RNA template, such as an analyte or an intermediate agent (e.g., a ligation product or an analyte capture agent), or a portion thereof), or derivatives thereof (see, e.g., Section (II) (b) (vii) of PCT Publication No. WO2020/176788 and/or U.S. Patent Application Publication No. 2020/0277663 regarding extended capture probes, which is herein incorporated by reference). In some cases, capture probes may be configured to form ligation products with a template (e.g., a DNA or RNA template, such as an analyte or an intermediate agent, or portion thereof), thereby creating ligation products that serve as proxies for the template.

As used herein, an “extended capture probe” refers to a capture probe having additional nucleotides added to a terminus (e.g., a 3′ or 5′ end) of the capture probe thereby extending the overall length of the capture probe. For example, an “extended 3′ end” indicates additional nucleotides were added to the most 3′ nucleotide of the capture probe to extend the length of the capture probe, for example, by polymerization reactions used to extend nucleic acid molecules including templated polymerization catalyzed by a polymerase (e.g., a DNA polymerase or a reverse transcriptase). In some embodiments, extending the capture probe includes adding to a 3′ end of a capture probe a nucleic acid sequence that is complementary to a nucleic acid sequence of an analyte or intermediate agent specifically bound to the capture domain of the capture probe. In some embodiments, the capture probe is extended by using a reverse transcriptase. In some embodiments, the capture probe is extended using one or more DNA polymerases. In some embodiments, the extended capture probes include the sequence of the capture domain, the sequence of the spatial barcode of the capture probe, and the complementary sequence of the template used for extension of the capture probe.

In some embodiments, extended capture probes are amplified (e.g., in bulk solution or on the array) to yield quantities that are sufficient for downstream analysis, e.g., sequencing. In some embodiments, extended capture probes (e.g., DNA molecules) can act as templates for an amplification reaction (e.g., a polymerase chain reaction).

Additional variants of spatial analysis methods, including in some embodiments, an imaging step, are described in Section (II) (a) of PCT Publication No. WO2020/176788 and/or U.S. Patent Application Publication No. 2020/0277663, which is herein incorporated by reference. Analysis of captured analytes (and/or intermediate agents or portions thereof), for example, including sample removal, extension of capture probes using the captured analyte as a template, sequencing (e.g., of a cleaved extended capture probe and/or a cDNA molecule complementary to an extended capture probe), sequencing on the array (e.g., using, for example, in situ hybridization or in situ ligation approaches), temporal analysis, and/or proximity capture, is described in Section (II) (g) of PCT Publication No. WO2020/176788 and/or U.S. Patent Application Publication No. 2020/0277663, which is herein incorporated by reference. Some quality control measures are described in Section (II) (h) of PCT Publication No. WO2020/176788 and/or U.S. Patent Application Publication No. 2020/0277663, which is herein incorporated by reference.

Spatial information can provide information of medical importance. For example, the methods described herein can allow for: identification of one or more biomarkers (e.g., diagnostic, prognostic, and/or for determination of efficacy of a treatment) of a disease or disorder; identification of a candidate drug target for treatment of a disease or disorder; identification (e.g., diagnosis) of a subject as having a disease or disorder; identification of stage and/or prognosis of a disease or disorder in a subject; identification of a subject as having an increased likelihood of developing a disease or disorder; monitoring of progression of a disease or disorder in a subject; determination of efficacy of a treatment of a disease or disorder in a subject; identification of a patient subpopulation for which a treatment is effective for a disease or disorder; modification of a treatment of a subject with a disease or disorder; selection of a subject for participation in a clinical trial; and/or selection of a treatment for a subject with a disease or disorder. Exemplary methods for identifying spatial information of biological and/or medical importance can be found in U.S. Patent Application Publication Nos. 2021/0140982, 2021/0198741, and 2021/0199660, each of which is herein incorporated by reference.

Spatial information can provide information of biological importance. For example, the methods described herein can allow for: identification of transcriptome and/or proteome expression profiles (e.g., in healthy and/or diseased tissue); identification of multiple analyte types in close proximity (e.g., nearest neighbor or proximity based analysis); determination of up-regulated and/or down-regulated genes and/or proteins in diseased tissue; characterization of tumor microenvironments; characterization of tumor immune responses; characterization of cells types and their co-localization in healthy and diseased tissue; and identification of genetic variants within tissues (e.g., based on gene and/or protein expression profiles associated with specific disease or disorder biomarkers).

For spatial array-based methods, a substrate may function as a support for direct or indirect attachment of capture probes to features of the array. A “feature” is an entity that acts as a support or repository for various molecular entities used in spatial analysis. In some embodiments, some or all of the features in an array are functionalized for analyte capture. Exemplary substrates are described in Section (II) (c) of PCT Publication No. WO2020/176788 and/or U.S. Patent Application Publication No. 2020/0277663, which is herein incorporated by reference. Exemplary features and geometric attributes of an array can be found in Sections (II) (d) (i), (II) (d) (iii), and (II) (d) (iv) of PCT Publication No. WO2020/176788 and/or U.S. Patent Application Publication No. 2020/0277663, which is herein incorporated by reference.

Generally, analytes and/or intermediate agents (or portions thereof) can be captured when contacting a biological sample with a substrate including capture probes (e.g., a substrate with capture probes embedded, spotted, printed, fabricated on the substrate, or a substrate with features (e.g., beads, wells) comprising capture probes). As used herein, “contact,” “contacted,” and/or “contacting,” a biological sample with a substrate refers to any contact (e.g., direct or indirect) such that capture probes can interact (e.g., bind covalently or non-covalently (e.g., hybridize)) with analytes from the biological sample. Capture can be achieved actively (e.g., using electrophoresis) or passively (e.g., using diffusion). Analyte capture is further described in Section (II) (c) of PCT Publication No. WO2020/176788 and/or U.S. Patent Application Publication No. 2020/0277663, which is herein incorporated by reference.

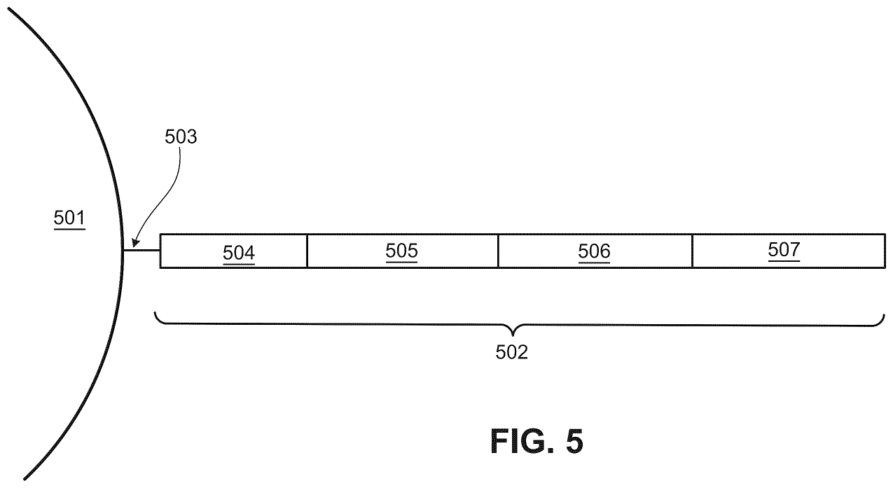

FIG. 5 is a schematic diagram showing an exemplary capture probe, as described herein. As shown, the capture probe 502 is optionally coupled to a feature 501 by a cleavage domain 503, such as a disulfide linker. The capture probe can include a functional sequence 504 that is useful for subsequent processing. The functional sequence 504 can include all or a part of sequencer specific flow cell attachment sequence (e.g., a P5 or P7 sequence), all or a part of a sequencing primer sequence, (e.g., a RI primer binding site, a R2 primer binding site), or a combination thereof. The capture probe can also include a spatial barcode 505. The capture probe can also include a unique molecular identifier (UMI) sequence 506. While FIG. 5 shows the spatial barcode 505 as being located upstream (5′) of UMI sequence 506, it is to be understood that capture probes wherein UMI sequence 506 is located upstream (5′) of the spatial barcode 505 is also suitable for use in any of the methods described herein. The capture probe can also include a capture domain 507 to facilitate capture of a target analyte. The capture domain can have a sequence complementary to a sequence of a nucleic acid analyte. The capture domain can have a sequence complementary to a connected probe described herein. The capture domain can have a sequence complementary to an analyte capture sequence present in an analyte capture agent. The capture domain can have a sequence complementary to a splint oligonucleotide. A splint oligonucleotide, in addition to having a sequence complementary to a capture domain of a capture probe, can have a sequence complementary to a sequence of a nucleic acid analyte, a portion of a connected probe described herein, a capture handle sequence described herein, and/or a methylated adaptor described herein.

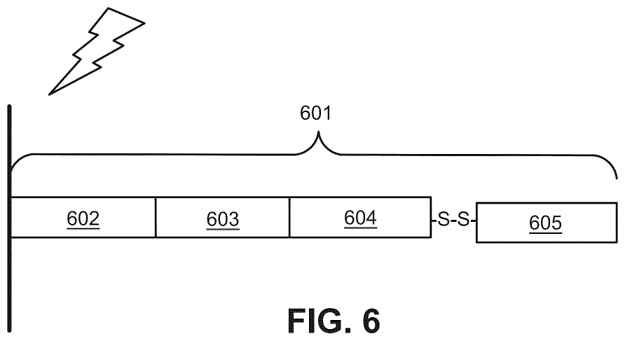

FIG. 6 is a schematic illustrating a cleavable capture probe, wherein the cleaved capture probe can enter into a non-permeabilized cell and bind to analytes within the cell. The capture probe 601 can contain a cleavage domain 602, a cell penetrating peptide 603, a reporter molecule 604, and a disulfide bond (—S—S—). 605 represents all other parts of a capture probe, for example a spatial barcode and a capture domain.

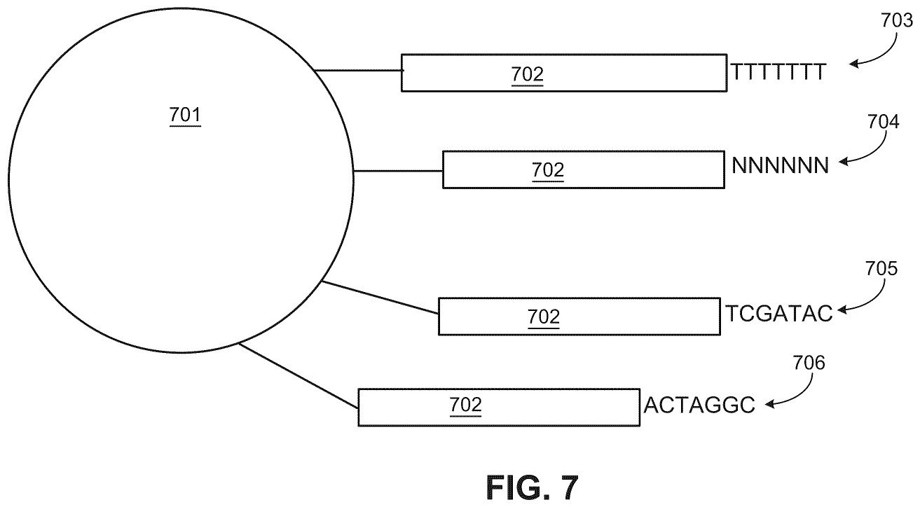

FIG. 7 is a schematic diagram of an exemplary multiplexed spatially-barcoded feature. In FIG. 7, the feature 701 can be coupled to spatially-barcoded capture probes, wherein the spatially-barcoded probes of a particular feature can possess the same spatial barcode, but have different capture domains designed to associate the spatial barcode of the feature with more than one target analyte. For example, a feature may include four different types of spatially-barcoded capture probes, each type of spatially-barcoded capture probe possessing the spatial barcode 702. One type of capture probe associated with the feature can include the spatial barcode 702 in combination with a poly (T) capture domain 703, designed to capture mRNA target analytes. A second type of capture probe associated with the feature can include the spatial barcode 702 in combination with a random N-mer capture domain 704 for gDNA analysis. A third type of capture probe associated with the feature can include the spatial barcode 702 in combination with a capture domain complementary to the analyte capture agent of interest 705. A fourth type of capture probe associated with the feature can include the spatial barcode 702 in combination with a capture probe that can specifically bind a nucleic acid molecule 706 that can function in a CRISPR assay (e.g., CRISPR/Cas9). While only four different capture probe-barcoded constructs are shown in FIG. 7, capture-probe barcoded constructs can be tailored for analyses of any given analyte associated with a nucleic acid and capable of binding with such a construct. For example, the schemes shown in FIG. 7 can also be used for concurrent analysis of other analytes disclosed herein, including, but not limited to: (a) mRNA, a lineage tracing construct, cell surface or intracellular proteins and/or metabolites, and gDNA; (b) mRNA, accessible chromatin (e.g., ATAC-seq, DNase-seq, and/or MNase-seq), cell surface or intracellular proteins and metabolites, and a perturbation agent (e.g., a CRISPR crRNA/sgRNA, TALEN, zinc finger nuclease, and/or antisense oligonucleotide as described herein); (c) mRNA, cell surface or intracellular proteins and/or metabolites, a barcoded labelling agent (e.g., the MHC multimers described herein), and a V (D) J sequence of an immune cell receptor (e.g., T-cell receptor). In some embodiments, a perturbation agent can be a small molecule, an antibody, a drug, an aptamer, a miRNA, a physical environmental (e.g., temperature) change, or any other known perturbation agents.

The functional sequences can generally be selected for compatibility with any of a variety of different sequencing systems, e.g., Ion Torrent Proton or PGM, Illumina sequencing instruments, PacBio, Oxford Nanopore, etc., and the requirements thereof. In some embodiments, functional sequences can be selected for compatibility with non-commercialized sequencing systems. Examples of such sequencing systems and techniques, for which suitable functional sequences can be used include, but are not limited to, Ion Torrent Proton or PGM sequencing, Illumina sequencing, PacBio SMRT sequencing, and Oxford Nanopore sequencing. Further, in some embodiments, functional sequences can be selected for compatibility with other sequencing systems, including non-commercialized sequencing systems.

In some embodiments, the spatial barcode 505 and functional sequences 504 are common to all of the probes attached to a given feature. In some embodiments, the UMI sequence 506 of a capture probe attached to a given feature is different from the UMI sequence of a different capture probe attached to the given feature.

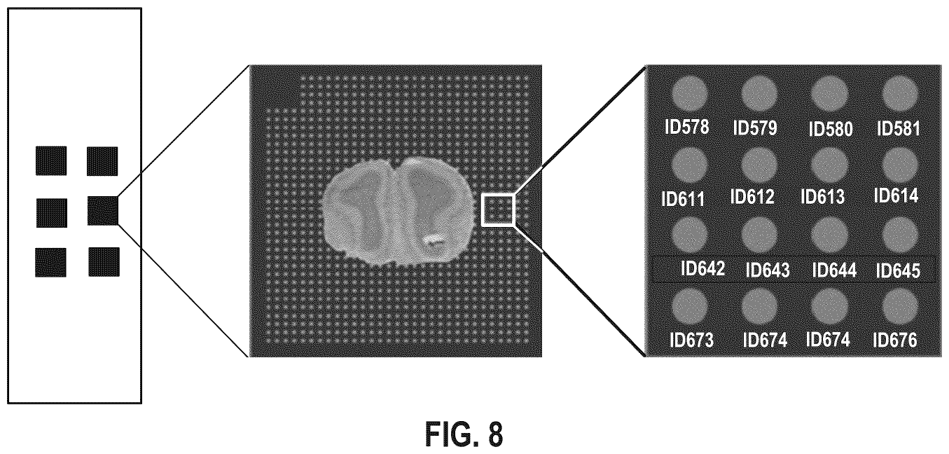

FIG. 8 depicts an exemplary arrangement of barcoded features within an array. From left to right, FIG. 8 shows (left) a slide including six spatially-barcoded arrays, (center) an enlarged schematic of one of the six spatially-barcoded arrays, showing a grid of barcoded features in relation to a biological sample, and (right) an enlarged schematic of one section of an array, showing the specific identification of multiple features within the array (e.g., labelled as ID578, ID579, ID580, etc.).

In some embodiments, more than one analyte type (e.g., nucleic acids and proteins) from a biological sample can be detected (e.g., simultaneously or sequentially) using any appropriate multiplexing technique, such as those described in Section (IV) of PCT Publication No. WO2020/176788 and/or U.S. Patent Application Publication No. 2020/0277663, which is herein incorporated by reference.

In some cases, spatial analysis can be performed by attaching and/or introducing a molecule (e.g., a peptide, a lipid, or a nucleic acid molecule) having a barcode (e.g., a spatial barcode) to a biological sample (e.g., to a cell in a biological sample). In some embodiments, a plurality of molecules (e.g., a plurality of nucleic acid molecules) having a plurality of barcodes (e.g., a plurality of spatial barcodes) are introduced to a biological sample (e.g., to a plurality of cells in a biological sample) for use in spatial analysis. In some embodiments, after attaching and/or introducing a molecule having a barcode to a biological sample, the biological sample can be physically separated (e.g., dissociated) into single cells or cell groups for analysis. Some such methods of spatial analysis are described in Section (III) of PCT Publication No. WO2020/176788 and/or U.S. Patent Application Publication No. 2020/0277663, which is herein incorporated by reference.

In some cases, spatial analysis can be performed by detecting multiple oligonucleotides that hybridize to an analyte. In some instances, for example, spatial analysis can be performed using RNA-templated ligation (RTL). Methods of RTL have been described previously. Sec, e.g., Credle et al., Nucleic Acids Res. 2017 Aug. 21; 45 (14): e128, which is herein incorporated by reference. Typically, RTL includes hybridization of two oligonucleotides to adjacent sequences on an analyte (e.g., an RNA molecule, such as an mRNA molecule). In some instances, the oligonucleotides are DNA molecules. In some instances, one of the oligonucleotides includes at least two ribonucleic acid bases at the 3′ end and/or the other oligonucleotide includes a phosphorylated nucleotide at the 5′ end. In some instances, one of the two oligonucleotides includes a capture domain (e.g., a poly (A) sequence or a non-homopolymeric sequence). After hybridization to the analyte, a ligase (e.g., a T4 RNA ligase (Rnl2), a PBCV-1 DNA Ligase or Chlorella virus DNA Ligase, a single-stranded DNA ligase, or a T4 DNA ligase) ligates the two oligonucleotides together, creating a ligation product. In some instances, the two oligonucleotides hybridize to sequences that are not adjacent to one another. For example, hybridization of the two oligonucleotides creates a gap between the hybridized oligonucleotides. In some instances, a polymerase (e.g., a DNA polymerase) can extend one of the oligonucleotides prior to ligation. After ligation, the ligation product is released from the analyte. In some instances, the ligation product is released using an endonuclease (e.g., RNase H). In some instances, the ligation product is removed using heat. In some instances, the ligation product is removed using potassium hydroxide (KOH). The released ligation product can then be captured by capture probes (e.g., instead of direct capture of an analyte) on an array, optionally amplified, and sequenced, thus determining the location and optionally the abundance of the analyte in the biological sample.

In some instances, one or both of the oligonucleotides may hybridize to genomic DNA (gDNA) which can lead to false positive sequencing data from ligation events on gDNA (off target) in addition to the desired (on target) ligation events on target nucleic acids (e.g., mRNA). Thus, in some embodiments, the disclosed methods can include contacting the biological sample with a deoxyribonuclease (DNase). The DNase can be an endonuclease or exonuclease. In some embodiments, the DNase digests single-stranded and/or double-stranded DNA. Suitable DNases include, without limitation, a DNase I and a DNase II. Use of a DNase as described can mitigate false positive sequencing data from off target gDNA ligation events.

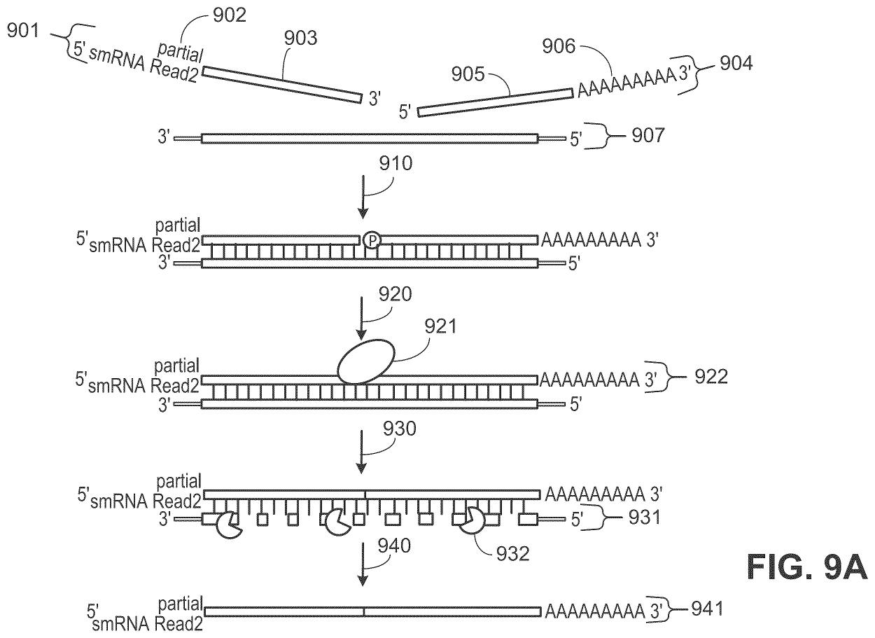

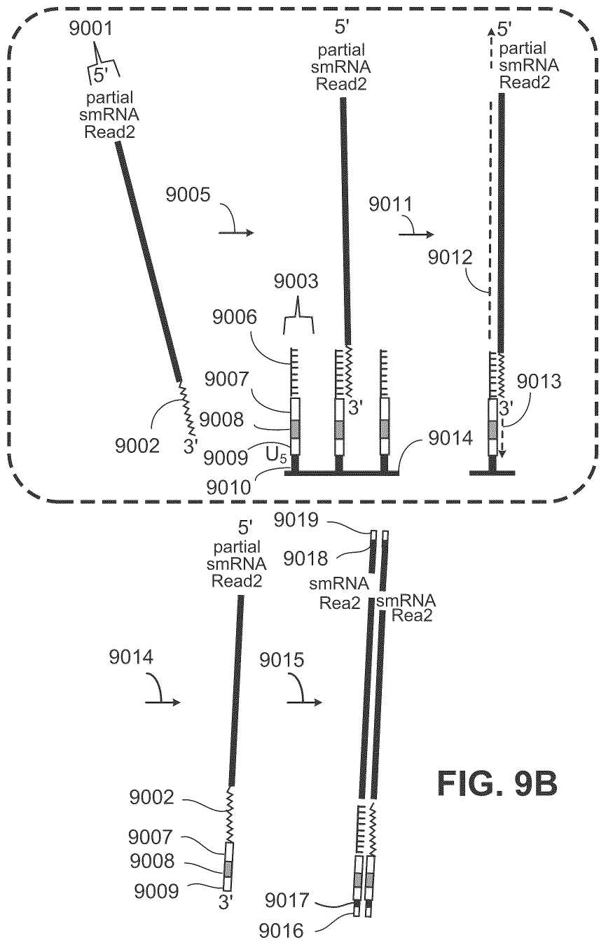

A non-limiting example of templated ligation methods disclosed herein is depicted in FIG. 9A. After a biological sample is contacted with a substrate including a plurality of capture probes and contacted with (a) a first probe 901 having a target-hybridization sequence 903 and a primer sequence 902 and (b) a second probe 904 having a target-hybridization sequence 905 and a capture domain (e.g., a poly (A) sequence) 906, the first probe 901 and the second probe 904 hybridize 910 to an analyte 907. A ligase 921 ligates 920 the first probe 901 to the second probe 904, thereby generating a ligation product 922. The ligation product 922 is then released 930 from the analyte 931 by digesting the analyte 907 using an endoribonuclease 932. The sample is permeabilized 940 and the ligation product 941 is able to hybridize to a capture probe on the substrate. Methods and composition for spatial detection using templated ligation have been described in PCT Publication No. WO 2021/133849 A1, U.S. Pat. Nos. 11,332,790 and 11,505,828, each of which is incorporated by reference in its entirety.

In some embodiments, as shown in FIG. 9B, the ligation product 9001 includes a capture probe capture domain 9002, which can bind to a capture probe 9003 (e.g., a capture probe immobilized, directly or indirectly, on a substrate 9004). In some embodiments, methods provided herein include contacting 9005 a biological sample with a substrate 9004, wherein the capture probe 9003 is affixed to the substrate (e.g., immobilized to the substrate, directly or indirectly). In some embodiments, the capture probe capture domain 9002 of the ligated product 9001 specifically binds to the capture domain 9006. The capture probe can also include a unique molecular identifier (UMI) 9007, a spatial barcode 9008, a functional sequence 9009, and a cleavage domain 9010.

In some embodiments, methods provided herein include permeabilization of the biological sample such that the capture probe can more easily bind to target analytes (i.e., compared to no permeabilization). In some embodiments, reverse transcription (RT) reagents can be added to permeabilize biological samples. Incubation with the RT reagents can be used to extend the capture probes 9011 to produce spatially-barcoded full-length cDNA 9012 and 9013 from the captured analytes (e.g., polyadenylated mRNA). Second strand reagents (e.g., second strand primers, enzymes, etc.) can be added to the biological sample to initiate second strand synthesis.

In some embodiments, methods provided herein include permeabilization of the biological sample such that the capture probe can more easily capture the ligation products (i.e., compared to no permeabilization). In some embodiments, reverse transcription (RT) reagents can be added to permeabilize biological samples. Incubation with the RT reagents can be used to extend the capture probes 9011 to produce spatially-barcoded full-length cDNA 9012 and 9013 from the captured ligation products (e.g., polyadenylated ligation products).

In some embodiments, the extended ligation products can be denatured 9014, released from the capture probe and transferred (e.g., to a clean tube) for amplification, and/or library construction. The spatially-barcoded ligation products can be amplified 9015 via PCR prior to library construction. P5 9016 and P7 9019 sequences can be used for sequencing, while i5 9017 and i7 9018 sequences can be used as sample indexes. The amplicons can then be sequenced using paired-end sequencing using TruSeq Read 1 and TruSeq Read 2 as sequencing primer sites.

In some embodiments, detection of one or more analytes (e.g., protein analytes) can be performed using one or more analyte capture agents. As used herein, an “analyte capture agent” refers to an agent that interacts with an analyte (e.g., an analyte in a biological sample) and with a capture probe (e.g., a capture probe attached to a substrate or a feature) to identify the analyte. In some embodiments, the analyte capture agent includes: (i) an analyte binding moiety (e.g., that binds to an analyte), for example, an antibody or antigen-binding fragment thereof; (ii) analyte binding moiety barcode; and (iii) an analyte capture sequence. As used herein, the term “analyte binding moiety barcode” refers to a barcode that is associated with or otherwise identifies the analyte binding moiety. As used herein, the term “analyte capture sequence” refers to a region or moiety configured to hybridize to, bind to, couple to, or otherwise interact with a capture domain of a capture probe. In some cases, an analyte binding moiety barcode (or portion thereof) may be able to be removed (e.g., cleaved) from the analyte capture agent. Additional description of analyte capture agents can be found in Section (II) (b) (ix) of PCT Publication No. WO2020/176788 and/or Section (II) (b) (viii) U.S. Patent Application Publication No. 2020/0277663, which is herein incorporated by reference.

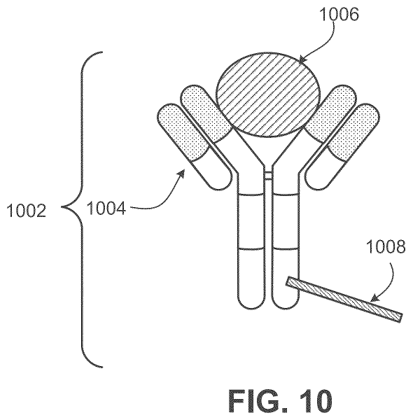

FIG. 10 is a schematic diagram of an exemplary analyte capture agent 1002 comprised of an analyte binding moiety 1004 and an analyte binding moiety barcode domain 1008. The exemplary analyte binding moiety 1004 is a molecule capable of binding to an analyte 1006 and the analyte capture agent 1002 is capable of interacting with a spatially-barcoded capture probe. The analyte binding moiety 1004 can bind to the analyte 1006 with high affinity and/or with high specificity. The analyte capture agent 1002 can include: (i) an analyte binding moiety barcode domain 1008 which serves to identify the analyte binding moiety, and (ii) an analyte capture sequence, which can hybridize to at least a portion or an entirety of a capture domain of a capture probe. The analyte binding moiety 1004 can include a polypeptide and/or an aptamer. The analyte binding moiety 1004 can include an antibody or antibody fragment (e.g., an antigen-binding fragment).

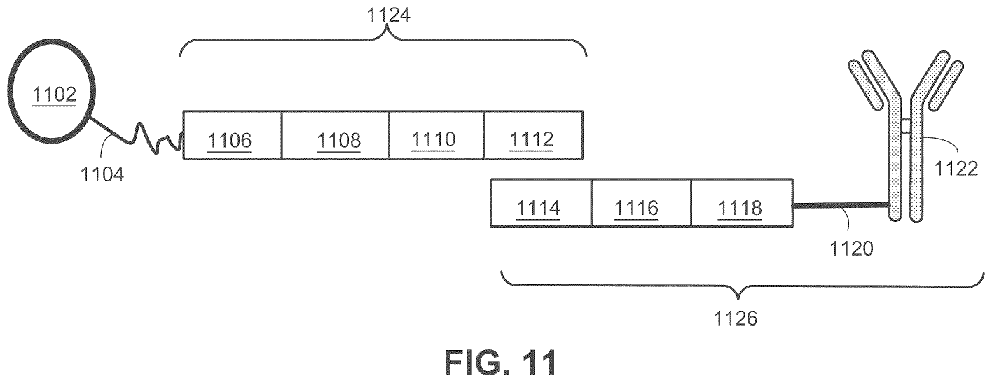

FIG. 11 is a schematic diagram depicting an exemplary interaction between a feature-immobilized capture probe 1124 and an analyte capture agent 1126. The feature-immobilized capture probe 1124 can include a spatial barcode 1108 as well as functional sequences 1106 and a UMI 1110, as described elsewhere herein. The capture probe can be affixed 1104 to a feature such as a bead 1102. The capture probe 1124 can also include a capture domain 1112 that is capable of binding to an analyte capture agent 1126. The analyte binding moiety barcode domain of the analyte capture agent 1126 can include a functional sequence 1118, analyte binding moiety barcode 1116, and an analyte capture sequence 1114 that is capable of binding (e.g., hybridizing) to the capture domain 1112 of the capture probe 1124. The analyte capture agent 1126 can also include a linker 1120 that allows the analyte binding moiety barcode domain (e.g., including the functional sequence 1118, analyte binding moiety barcode 1116, and analyte capture sequence 1114) to couple to the analyte binding moiety 1122. In some embodiments, the linker 1120 is a cleavable linker. In some embodiments, the cleavable linker is a photo-cleavable linker, a UV-cleavable linker, chemical-cleavable linker, thermal-cleavable linker, or an enzyme cleavable linker. In some instances, the cleavable linker is a disulfide linker. A disulfide linker can be cleaved by use of a reducing agent, such as dithiothreitol (DTT), beta-mercaptoethanol (BME), or tris(2-carboxyethyl) phosphine (TCEP).

During analysis of spatial information, sequence information for a spatial barcode associated with an analyte is obtained, and the sequence information can be used to provide information about the spatial distribution of the analyte in the biological sample. Various methods can be used to obtain the spatial information. In some embodiments, specific capture probes and the analytes they capture are associated with specific locations in an array of features on a substrate. For example, specific spatial barcodes can be associated with specific array locations prior to array fabrication, and the sequences of the spatial barcodes can be stored (e.g., in a database) along with specific array location information, so that each spatial barcode uniquely maps to a particular array location.

Alternatively, specific spatial barcodes can be deposited at predetermined locations in an array of features during fabrication such that at each location, only one type of spatial barcode is present so that each spatial barcode is uniquely associated with a single feature of the array. Where necessary, the arrays can be decoded using any of the methods described herein so that spatial barcodes are uniquely associated with array feature locations, and this mapping can be stored as described above.

When sequence information is obtained for capture probes and/or analytes during analysis of spatial information, the locations of the capture probes and/or analytes can be determined by referring to the stored information that uniquely associates each spatial barcode with an array feature location. In this manner, specific capture probes and captured analytes are associated with specific locations in the array of features. Each array feature location represents a position relative to a coordinate reference point (e.g., an array location, a fiducial marker) for the array. Accordingly, each feature location has an “address” or location in the coordinate space of the array.

Some exemplary spatial analysis workflows are described in the Exemplary Embodiments section of PCT Publication No. WO2020/176788 and/or U.S. Patent Application Publication No. 2020/0277663, which is herein incorporated by reference. See, for example, the Exemplary embodiment starting with “In some non-limiting examples of the workflows described herein, the sample can be immersed . . . ” of PCT Publication No. WO2020/176788 and/or U.S. Patent Application Publication No. 2020/0277663, which is herein incorporated by reference. See also, e.g., the Visium Spatial Gene Expression Reagent Kits User Guide (e.g., Rev F, dated January 2022); and/or the Visium Spatial Gene Expression Reagent Kits-Tissue Optimization User Guide (e.g., Rev E, dated February 2022), each of which is herein incorporated by reference in its entirety.

In some embodiments, spatial analysis can be performed using dedicated hardware and/or software, such as any of the systems described in Sections (II) (e) (ii) and/or (V) of PCT Publication No. WO2020/176788 and/or U.S. Patent Application Publication No. 2020/0277663, or any of one or more of the devices or methods described in Sections Control Slide for Imaging, Methods of Using Control Slides and Substrates for, Systems of Using Control Slides and Substrates for Imaging, and/or Sample and Array Alignment Devices and Methods, Informational labels of PCT Publication No. WO2020/123320, which is herein incorporated by reference.

Suitable systems for performing spatial analysis can include components such as a chamber (e.g., a flow cell or a scalable, fluid-tight chamber) for containing a biological sample. The biological sample can be mounted, for example, in a biological sample holder. One or more fluid chambers can be connected to the chamber and/or the sample holder via fluid conduits, and fluids can be delivered into the chamber and/or sample holder via fluidic pumps, vacuum sources, or other devices coupled to the fluid conduits that create a pressure gradient to drive fluid flow. One or more valves can also be connected to fluid conduits to regulate the flow of reagents from reservoirs to the chamber and/or sample holder.

The systems can optionally include a control unit that includes one or more electronic processors, an input interface, an output interface (such as a display), and a storage unit (e.g., a solid state storage medium such as, but not limited to, a magnetic, optical, or other solid state, persistent, writeable, and/or re-writeable storage medium). The control unit can optionally be connected to one or more remote devices via a network. The control unit (and components thereof) can generally perform any of the steps and functions described herein. Where the system is connected to a remote device, the remote device (or devices) can perform any of the steps or features described herein. The systems can optionally include one or more detectors (e.g., CCD or CMOS) used to capture images. The systems can also optionally include one or more light sources (e.g., LED-based, diode-based, or lasers) for illuminating a sample, a substrate with features, analytes from a biological sample captured on a substrate, and various control and calibration media.

The systems can optionally include software instructions encoded and/or implemented in one or more of tangible storage media and hardware components such as application specific integrated circuits. The software instructions, when executed by a control unit (and in particular, an electronic processor) or an integrated circuit, can cause the control unit, integrated circuit, or other component executing the software instructions to perform any of the method steps or functions described herein.

In some cases, the systems described herein can detect (e.g., register an image) the biological sample on the array. Exemplary methods to detect the biological sample on an array are described in PCT Publication No. WO2021/102003 and/or U.S. Patent Application Publication No. 2021/0150707, each of which is incorporated herein by reference in its entirety.

Prior to transferring analytes from the biological sample to the array of features on the substrate, the biological sample can be aligned with the array. Alignment of a biological sample and an array of features including capture probes can facilitate spatial analysis, which can be used to detect differences in analyte presence and/or level within different positions in the biological sample, for example, to generate a three-dimensional map of the analyte presence and/or level. Exemplary methods to generate a two-dimensional and/or three-dimensional map of the analyte presence and/or level are described in PCT Publication No. WO2020/053655 and spatial analysis methods are generally described in PCT Publication No. WO2021/102039 and/or U.S. Patent Application Publication No. 2021/0155982, each of which is incorporated herein by reference in their entireties.

In some cases, a map of analyte presence and/or level can be aligned to an image of a biological sample using one or more fiducial markers, e.g., objects placed in the field of view of an imaging system which appear in the image produced, as described in the Substrate Attributes Section, Control Slide for Imaging Section of PCT Publication Nos. WO2020/123320, WO 2021/102005, and/or U.S. Patent Application Publication No. 2021/0158522, each of which is incorporated herein by reference in its entirety. Fiducial markers can be used as a point of reference or measurement scale for alignment (e.g., to align a sample and an array, to align two substrates, to determine a location of a sample or array on a substrate relative to a fiducial marker) and/or for quantitative measurements of sizes and/or distances.

### B. Reusable Spatial Arrays Determining the Location of an Analyte in a Biological Sample

### (i) Introduction

The present disclosure features reusable spatial arrays for determining the spatial location of an analyte based on the use of removable spatially barcoded capture probes. In some embodiments, the removable barcoded capture probes are released through different release mechanisms. In some embodiments, the release mechanism includes a cleavable linker. In some embodiments, more than one set of capture probes is on the array (e.g., two sets, three sets, four sets, or more). In some embodiments, each capture probe set includes a different release mechanism. In some embodiments, the reusable arrays are reused to capture additional analytes from the sample biological sample. In some embodiments, the reusable arrays are reused to capture analytes from a second biological sample.

Cleavable linkers are known in the art, of which examples include, but are not limited to, linkers that are sensitive to one or more enzymes, pH, temperature, light, shear stress, sonication, a chemical agent (e.g., dithiothreitol), or any combination thereof. In some embodiments, the cleavable linker can be sensitive to light or enzyme activity (e.g., a restriction endonuclease).

### (ii) Methods of Using Reusable Arrays

Provided herein are methods for determining the spatial location of an analyte in a biological sample, the method including: (a) disposing the biological onto a first substrate; (b) providing a second substrate including an array, where the array includes a first set of capture probes and a second set of capture probes, where a first capture probe of the first set of capture probes includes: (i) a first spatial barcode; (ii) a first capture domain; and (iii) a first release mechanism (e.g., a first restriction endonuclease cleavage site, a first photocleavable site, etc.); and where a second capture probe of the second set of capture probes includes: (i) a second spatial barcode; (ii) a second capture domain; and (iii) a second release mechanism (e.g., a second restriction endonuclease cleavage site, a second photocleavable site, etc.); where the first release mechanism differs from the second release mechanism (e.g., the sequence of the first restriction endonuclease cleavage site of the first capture probe differs from the sequence of the second restriction endonuclease cleavage site of the second capture probe); (c) hybridizing the analyte to the first capture domain; (d) releasing the first set of capture probes from the array; and (e) determining (i) all or a part of the sequence of the analyte, or a complement thereof, and (ii) the sequence of the first spatial barcode, or a complement thereof, and using the determined sequences of (i) and (ii) to determine the spatial location of the analyte in the biological sample.

Also provided herein are methods for determining the spatial location of an analyte in a biological sample, the method including: (a) disposing the biological onto a first substrate; (b) hybridizing a first probe and a second probe to the analyte, where the first probe and the second probe each include a sequence that is substantially complementary to sequences of the analyte, and where the second probe includes a capture probe binding domain; (c) coupling the first probe and the second probe, thereby generating a ligation product; (d) providing a second substrate including an array, where the array includes a first set of capture probes and a second set of capture probes, where a first capture probe of the first set of capture probes includes: (i) a first spatial barcode; (ii) a first capture domain; and (iii) a first release mechanism (e.g., a first restriction endonuclease cleavage site, a first photocleavable site, etc.) and where a second capture probe of the second set of capture probes includes: (i) a second spatial barcode; (ii) a second capture domain; and (iii) a second release mechanism (e.g., a second restriction endonuclease cleavage site, a second photocleavable site, etc.); where the first release mechanism differs from the second release mechanism (e.g., the sequence of the first restriction endonuclease cleavage site of the first capture probe differs from the sequence of the second restriction endonuclease cleavage site of the second capture probe); (c) hybridizing the ligation product to the first capture domain of the first capture probe; (f) releasing the first set of capture probes from the array; and (g) determining (i) all or a part of the sequence of the analyte or a complement thereof or the ligation product, or a complement thereof, and (ii) the sequence of the first spatial barcode, or a complement thereof, and using the determined sequences of (i) and (ii) to determine the spatial location of the analyte in the biological sample.

Also provided herein are methods for determining the spatial location of an analyte in a biological sample, the method including: (a) contacting the biological sample with an array, where the array includes a first set of capture probes and a second set of capture probes, where a first capture probe of the first set of capture probes includes: (i) a first spatial barcode; (ii) a first capture domain; and (iii) a first restriction endonuclease cleavage site; and where a second capture probe of the second set of capture probes includes: (i) a second spatial barcode; (ii) a second capture domain; and (iii) a second restriction endonuclease cleavage site; where the sequence of the first restriction endonuclease cleavage site of the first capture probe differs from the sequence of the second restriction endonuclease cleavage site of the second capture probe; (b) hybridizing the analyte to the first capture domain; (c) releasing the first set of capture probes from the array; and (d) determining (i) all or a part of the sequence of the analyte, or a complement thereof, and (ii) the sequence of the first spatial barcode, or a complement thereof, and using the determined sequences of (i) and (ii) to determine the spatial location of the analyte in the biological sample.

Also provided herein are methods for determining the spatial location of an analyte in a biological sample, the method including: (a) contacting the biological sample with an array, where the array includes a first set of capture probes and a second set of capture probes, where a first capture probe of the first set of capture probes includes: (i) a first spatial barcode; (ii) a first capture domain; and (iii) a first release mechanism (e.g., a first restriction endonuclease cleavage site, a first photocleavable site, etc.); and where a second capture probe of the second set of capture probes includes: (i) a second spatial barcode; (ii) a second capture domain; and (iii) a second release mechanism (e.g., a second restriction endonuclease cleavage site, a second photocleavable site, etc.); where the first release mechanism differs from the second release mechanism (e.g., the sequence of the first restriction endonuclease cleavage site of the first capture probe differs from the sequence of the second restriction endonuclease cleavage site of the second capture probe); (b) hybridizing the analyte to the first capture domain; (c) releasing the first set of capture probes from the array; and (d) determining (i) all or a part of the sequence of the analyte, or a complement thereof, and (ii) the sequence of the first spatial barcode, or a complement thereof, and using the determined sequences of (i) and (ii) to determine the spatial location of the analyte in the biological sample.

Also provided herein are methods for determining the spatial location of an analyte in a biological sample, the method including: (a) contacting the biological sample with an array, where the array includes a first set of capture probes and a second set of capture probes, where a first capture probe of the first set of capture probes includes: (i) a first spatial barcode; (ii) a first capture domain; and (iii) a first release mechanism (e.g., a first restriction endonuclease cleavage site); and where a second capture probe of the second set of capture probes includes: (i) a second spatial barcode; (ii) a second capture domain; and (iii) a second release mechanism (e.g., a second restriction endonuclease cleavage site); where the first release mechanism differs from the second release mechanism (e.g., the sequence of the first restriction endonuclease cleavage site of the first capture probe differs from the sequence of the second restriction endonuclease cleavage site of the second capture probe); (b) hybridizing a first probe and a second probe to the analyte, where the first probe and the second probe each include a sequence that is substantially complementary to sequences of the analyte, and where the second probe includes a capture probe binding domain; (c) coupling the first probe and the second probe, thereby generating a ligation product; (d) hybridizing the ligation product to the first capture domain of the first capture probe; (c) releasing the first set of capture probes from the array; and (f) determining (i) all or a part of the sequence of the analyte or a complement thereof or the ligation product, or a complement thereof, and (ii) the sequence of the first spatial barcode, or a complement thereof, and using the determined sequences of (i) and (ii) to determine the spatial location of the analyte in the biological sample.

Any suitable restriction endonuclease cleavage site (i.e. restriction site) can be used in the methods described herein. Cleavage methods and procedures for selecting restriction enzymes for cutting nucleic acid at specific sites are well known to the skilled artisan. For example, many suppliers of restriction enzymes provide information on conditions and types of DNA sequences cut by specific restriction enzymes, including New England BioLabs, Pro-Mega Biochems, Bochringer-Mannheim, and the like.

Restriction enzymes (i.e., restriction endonucleases) are traditionally classified into three types on the basis of subunit composition, cleavage position, sequence-specificity and cofactor-requirements. However, amino acid sequencing has uncovered extraordinary variety among restriction enzymes and revealed that at the molecular level there are many more than three different kinds.

Type I enzymes are complex, multi-subunit, combination restriction-and-modification enzymes that cut DNA at random far from their recognition sequences. Type I enzymes do not produce discrete restriction fragments or distinct gel-banding patterns.

Type II enzymes cut DNA at defined positions close to or within their recognition sequences. They produce discrete restriction fragments and distinct gel banding patterns

The most common type II enzymes are those like HhaI, HindIII and NotI that cleave DNA within their recognition sequences. Enzymes of this kind are available commercially. Most recognize DNA sequences that are symmetric because they bind to DNA as homodimers, but a few, (e.g., BbvCI: CCTCAGC) recognize asymmetric DNA sequences because they bind as heterodimers. Some enzymes recognize continuous sequences (e.g., EcoRI: GAATTC) in which the two half-sites of the recognition sequence are adjacent, while others recognize discontinuous sequences in which the half-sites are separated. Cleavage leaves a 3′-hydroxyl on one side of each cut and a 5′-phosphate on the other. They require only magnesium for activity and the corresponding modification enzymes require only S-adenosylmethionine. They tend to be small, with subunits in the 200-350 amino acid range.

The next most common type II enzymes, usually referred to as ‘type IIs’ are those like FokI and AlwI that cleave outside of their recognition sequence to one side. These enzymes are intermediate in size, 400-650 amino acids in length, and they recognize sequences that are continuous and asymmetric. They comprise two distinct domains, one for DNA binding and the other for DNA cleavage. They are thought to bind to DNA as monomers for the most part, but to cleave DNA cooperatively, through dimerization of the cleavage domains of adjacent enzyme molecules. For this reason, some type IIs enzymes are much more active on DNA molecules that contain multiple recognition sites. A wide variety of Type IIS restriction enzymes are known and such enzymes have been isolated from bacteria, phage, archaebacterial and viruses of eukaryotic algae and are commercially available (Promega, Madison Wis.; New England Biolabs, Beverly, Mass.). Examples of Type LIS restriction enzymes that may be used with methods described herein include, but are not limited to enzymes such as those listed in Table 1.

TABLE 1
Examples of Type IIS restriction enzymes

| | Recognition/ | |
|---|---|---|
| Enzyme-Source | Cleavage site | Supplier |
| AlwI-Acinetobacter lwoffii | GGATC(4/5) | NE Biolabs |
| Alw26I-Acinetobacter lwoffi | GTCTC(1/5) | Promega |
| BbsI-Bacillus laterosporus | GAAGAC(2/6) | NE Biolabs |
| BbvI-Bacillus brevis | GCAGC(8/12) | NE Biolabs |
| BceAI-Bacillus cereus 1315 | IACGGC(12/14) | NE Biolabs |
| BmrI-Bacillus megaterium | CTGGG(5/4) | NE Biolabs |
| BsaI-Bacillus | GGTCTC(1/5) | NE Biolabs |
| stearothermophilus 6-55 | | |
| Bst71I-Bacillus | GCAGC(8/12) | Promega |
| stearothermophilus 71 | | |
| BsmAI-Bacillus | GTCTC(1/5) | NE Biolabs |
| stearothermophilus A664 | | |
| BsmBI-Bacillus | CGTCTC(1/5) | NE Biolabs |
| stearothermophilus B61 | | |
| BsmFI-Bacillus | GGGAC(10/14) | NE Biolabs |
| stearothermophilus F | | |
| BspMI-Bacillus species M | ACCTGC(4/8) | NE Biolabs |
| EarI-Enterobacter aerogenes | CTCTTC(1/4) | NE Biolabs |
| FauI-Flavobacterium aquatile | CCCGC(4/6) | NE Biolabs |
| FokI-Flavobacterium | GGATG(9/13) | NE Biolabs |
| okeonokoites | | |
| HgaI-Haemophilus gallinarum | GACGC(5/10) | NE Biolabs |
| PleI-Pseudomonas lemoignei | GAGTC(4/5) | NE Biolabs |
| SapI-Saccharopolyspora species | GCTCTTC(1/4) | NE Biolabs |
| SfaNI-Streptococcus | GCATC(5/9) | NE Biolabs |
| faecalis ND547 | | |
| Sth132I-Streptococcus | CCCG(4/8) | Gene |
| thermophilus ST132 | | 195: 201-206 (1997) |

A third major kind of type II enzyme, more properly referred to as “type IV” are large, combination restriction-and-modification enzymes, 850-1250 amino acids in length, in which the two enzymatic activities reside in the same protein chain. These enzymes cleave outside of their recognition sequences; those that recognize continuous sequences (e.g., Eco57I: CTGAAG) cleave on just one side; those that recognize discontinuous sequences cleave on both sides releasing a small fragment containing the recognition sequence. The amino acid sequences of these enzymes are varied but their organization are consistent. They comprise an N-terminal DNA-cleavage domain joined to a DNA-modification domain and one or two DNA sequence-specificity domains forming the C-terminus, or present as a separate subunit. When these enzymes bind to their substrates, they switch into either restriction mode to cleave the DNA, or modification mode to methylate it.

As discussed above, the length of restriction recognition sites varies. For example, the enzymes EcoRI, SacI and SstI each recognize a 6 base-pair (bp) sequence of DNA, whereas NotI recognizes a sequence 8 bp in length, and the recognition site for Sau3AI is only 4 bp in length. Length of the recognition sequence dictates how frequently the enzyme will cut in a random sequence of DNA. Enzymes with a 6 bp recognition site will cut, on average, every 46 or 4096 bp; a 4 bp recognition site will occur roughly every 256 bp.

Different restriction enzymes can have the same recognition site-such enzymes are called isoschizomers. For example, the recognition sites for SacI and SstI are identical. In some cases isoschizomers cut identically within their recognition site, but sometimes they do not. Isoschizomers often have different optimum reaction conditions, stabilities and costs, which may influence the decision of which to use.

Restriction recognition sites can be unambiguous or ambiguous. The enzyme BamHI recognizes the sequence GGATCC and no others; therefore it is considered “unambiguous.” In contrast, HinfI recognizes a 5 bp sequence starting with GA, ending in TC, and having any base between. HinfI has an ambiguous recognition site. XholI also has an ambiguous recognition site and will recognize and cut sequences of AGATCT, AGATCC, GGATCT and GGATCC.

The recognition site for one enzyme may contain the restriction site for another. For example, note that a BamHI recognition site contains the recognition site for Sau3AI. Consequently, all BamHI sites will cut with Sau3AI. Similarly, one of the four possible XholI sites will also be a recognition site for BamHI and all four will cut with Sau3AI.

Most recognition sequences are palindromes-they read the same forward (5′ to 3′ on the top strand) and backward (5′ to 3′ on the bottom strand). Most, but certainly not all recognition sites for commonly-used restriction enzymes are palindromes. Most restriction enzymes bind to their recognition site as dimers (pairs).

In some embodiments, the first release mechanism is 5′ to the first spatial barcode and the first capture domain of the first capture probe. In some embodiments, the first restriction endonuclease cleavage site is 5′ to the first spatial barcode and the first capture domain of the first capture probe. In some embodiments, the second restriction endonuclease cleavage site is 5′ to the second spatial barcode and the second capture domain of the second capture probe. In some embodiments, the second release mechanism is 5′ to the second spatial barcode and the second capture domain of the second capture probe.

In some embodiments, the array comprises a third set, a fourth set, or a fifth set of capture probes. In some embodiments, the array comprises about 1, about 2, about 3, about 4, about 5, about 6, about 7, about 8, about 9 sets or more of capture probes. In some embodiments, each set of capture probes includes a unique release mechanism (e.g., restriction endonuclease cleavage site, photocleavable site, etc.). As used herein in the context of capture probes, “release mechanism” refers to a feature of a capture probe that permits its release or separation from the array. Such feature can be, but is not limited to, a nucleotide, a moiety, a bond, a sequence, or a domain that is part of the capture probe.

In some embodiments, the array comprises one or more sets of capture probes at a feature (e.g., at each feature) of the array. For example, an array may include 1, 2, 3, 4, 5, 6 or more sets of capture probes attached at each well, bead, spot, etc. of the array. As one set of capture probes is released from the array (e.g., due to its unique release mechanism), the other sets of capture probes remain and are available for capture of further analytes or analyte intermediates. In some embodiments, an array includes a plurality of beads. For example, an array can include a monolayer of beads where each bead occupies a unique position on a substrate comprising the array. In some instances, the beads can be immobilized on the substrate. The beads in the array can each contain a plurality of capture probes. In some instances, the capture probes on a particular bead have the same barcode, which is unique, and thus differs from the barcodes of capture probes on other beads in the array. Thus, the barcode contained by the capture probes on each bead can serve as a spatial barcode that is associated with a distinct position on the array. In some embodiments, an array includes 1, 2, 3, 4, 5, 6 or more sets of capture probes attached at each bead of a plurality of beads in the array.

In some embodiments, the array is a reusable array because different sets of capture probes include different release mechanisms (e.g., restriction endonuclease cleavage sites) and each use of the array releases one set of capture probes that include a specific release mechanism (e.g., restriction endonuclease cleavage site). The amount of reuse can be based on the number of different types of release mechanisms (e.g., restriction endonuclease cleavage sites) that are included in the capture probes on each array.

In some embodiments, the sequence of the first restriction endonuclease cleavage site and/or the sequence of the second restriction endonuclease site is about 5 bp, about 6 bp, about 7 bp, about 9 bp, about 9 bp, about 10 bp, about 11 bp, about 12 bp, about 13 bp, about 14 bp, about 15 bp or more in length. In some embodiments, the sequence of the first restriction endonuclease cleavage site and/or the sequence of the second restriction endonuclease site is about 7 bp or about 8 bp in length.

Also provided herein are methods for determining the spatial location of an analyte in a biological sample, the method including: (a) disposing the biological onto a first substrate; (b) providing a second substrate including an array, where the array includes a first set of capture probes and a second set of capture probes, where a first capture probe of the first set of capture probes includes: (i) a first spatial barcode; (ii) a first capture domain; and (iii) a first photocleavable cleavage site; and where a second capture probe of the second set of capture probes includes: (i) a second spatial barcode; (ii) a second capture domain; and (iii) a second photocleavable cleavage site; where the first photocleavable cleavage site of the first capture probe differs from the second photocleavable cleavage site of the second capture probe; (c) hybridizing the analyte to the first capture domain; (d) releasing the first set of capture probes from the array; and (c) determining (i) all or a part of the sequence of the analyte, or a complement thereof, and (ii) the sequence of the first spatial barcode, or a complement thereof, and using the determined sequences of (i) and (ii) to determine the spatial location of the analyte in the biological sample.

Also provided herein are methods for determining the spatial location of an analyte in a biological sample, the method including: (a) disposing the biological sample onto a first substrate; (b) hybridizing a first probe and a second probe to the analyte, where the first probe and the second probe each include a sequence that is substantially complementary to adjacent sequences of the analyte, and where the second probe includes a capture probe binding domain; (c) coupling the first probe and the second probe, thereby generating a ligation product; (d) providing a second substrate including an array, where the array includes a first set of capture probes and a second set of capture probes, where a first capture probe of the first set of capture probes includes: (i) a first spatial barcode; (ii) a first capture domain; and (iii) a first photocleavable cleavage site; and where a second capture probe of the second set of capture probes includes: (i) a second spatial barcode; (ii) a second capture domain; and (iii) a second photocleavable cleavage site; where the first photocleavable site of the first capture probe differs from the second photocleavable site of the second capture probe; (c) hybridizing the ligation product to the first capture domain of the first capture probe; (f) releasing the first set of capture probes from the array; and (g) determining (i) all or a part of the sequence of the analyte or ligation product, or a complement thereof, and (ii) the sequence of the first spatial barcode, or a complement thereof, and using the determined sequences of (i) and (ii) to determine the spatial location of the analyte in the biological sample.

Also provided herein are methods for determining the spatial location of an analyte in a biological sample, the method including: (a) contacting the biological with an array, where the array includes a first set of capture probes and a second set of capture probes, where a first capture probe of the first set of capture probes includes: (i) a first spatial barcode; (ii) a first capture domain; and (iii) a first photocleavable cleavage site; and where a second capture probe of the second set of capture probes includes: (i) a second spatial barcode; (ii) a second capture domain; and (iii) a second photocleavable cleavage site; where the first photocleavable cleavage site of the first capture probe differs from the second photocleavable cleavage site of the second capture probe; (b) hybridizing the analyte to the first capture domain; (c) releasing the first set of capture probes from the array; and (d) determining (i) all or a part of the sequence of the analyte, or a complement thereof, and (ii) the sequence of the first spatial barcode, or a complement thereof, and using the determined sequences of (i) and (ii) to determine the spatial location of the analyte in the biological sample.

Also provided herein are methods for determining the spatial location of an analyte in a biological sample, the method including: (a) contacting the biological sample with an array, where the array includes a first set of capture probes and a second set of capture probes, where a first capture probe of the first set of capture probes includes: (i) a first spatial barcode; (ii) a first capture domain; and (iii) a first photocleavable cleavage site; and where a second capture probe of the second set of capture probes includes: (i) a second spatial barcode; (ii) a second capture domain; and (iii) a second photocleavable cleavage site; where the first photocleavable site of the first capture probe differs from the second photocleavable site of the second capture probe; (b) hybridizing a first probe and a second probe to the analyte, where the first probe and the second probe each include a sequence that is substantially complementary to adjacent sequences of the analyte, and where the second probe includes a capture probe binding domain; (c) coupling the first probe and the second probe, thereby generating a ligation product; (d) hybridizing the ligation product to the first capture domain of the first capture probe; (e) releasing the first set of capture probes from the array; and (f) determining (i) all or a part of the sequence of the analyte or ligation product, or a complement thereof, and (ii) the sequence of the first spatial barcode, or a complement thereof, and using the determined sequences of (i) and (ii) to determine the spatial location of the analyte in the biological sample. As used herein, the term “capture probe binding domain” refers to a region or moiety configured to hybridize to, bind to, couple to, or otherwise interact with a capture domain of a capture probe.

In some embodiments, the first photocleavable cleavage site is 5′ to the first spatial barcode and the first capture domain of the first capture probe, and/or where and the second photocleavable cleavage site is 5′ to the second spatial barcode and the second capture domain of the second capture probe.

In some embodiments, the array comprises a third set, a fourth set, or a fifth set of capture probes. In some embodiments, the array comprises about 1, about 2, about 3, about 4, about 5, about 6, about 7, about 8, about 9 sets or more of capture probes. In some embodiments, each set of capture probes includes a unique release mechanism.

In some embodiments, the array is a reusable array because different sets of capture probes include different photocleavable cleavage sites and each use of the array releases one set of capture probes that include a specific photocleavable cleavage site. The amount of reuse can be based on the number of different types of photocleavable cleavage sites that are used in the capture probes on each array. For example, the array can be reused to capture additional analytes from the same biological sample. Additionally and/or alternatively, the array can be reused to capture analytes from a second, a third, a fourth, a fifth, a sixth, a seventh, an eighth, a ninth biological sample or more.

Any suitable photocleavable cleavage site known in the art can be used in the methods described herein. Cleavable linkers are useful in many oligonucleotide applications. Photo-cleavable linkers are chemically more stable than some other types of cleavable linker such as disulfide linkers therefore the cleavage reaction is more controlled. Any art-recognized photocleavable linker (i.e., photocleavable cleavage site) can be used for the methods described herein. Exemplary photocleavable linkers are provided in, e.g., PCT Publication No. WO2014200767A1, which is hereby incorporated by reference.

In some embodiments, a photocleavable linker includes a connecting or coupling point that connects, directly or indirectly, to a capture probe (e.g., on one or more nucleosides of the capture probe) described herein. The connecting point can be a bond, or comprise an atom, a molecule, and/or a linker described herein. In some preferred embodiments, the connecting point is a bond.

In some embodiments where a photocleavable linker is used, the capture probe can be released from the array by exposing the array to a light of a specified wavelength. In some embodiments, ultraviolet light is used to release the capture probe from the array.

In some embodiments, the releasing of the first set of capture probes includes exposing the array to light, e.g., UV light. In some embodiments, the method includes releasing the second set of capture probes, where the first photocleavable cleavage site and the second photocleavable cleavage site are cleaved by exposure to different wavelengths of light. Thus, in some embodiments, the methods include exposing the array to multiple (e.g., 2, 3, 4, 5, 6 or more) different wavelengths of light. In some embodiments, light having a wavelength of about 100-400 nanometers (nm) is used to release the capture probe(s) from the array. In some embodiments, light having a wavelength of about 100-280 nm, 280-315 nm, or 315-400 nm is used to release the capture probe(s) from the array.

In some embodiments, releasing of the first set of capture probes is irreversible.

In some embodiments, releasing the first set of capture probes from the array is performed after the analyte hybridizes to the first capture domain. In some embodiments, hybridizing the analyte to the first capture domain is performed after releasing the first set of capture probes from the array.

In some embodiments, the method includes hybridizing a second analyte in the biological sample or an analyte from a second biological sample to the second capture domain. In some embodiments, hybridizing the second analyte to the second capture domain is performed before releasing the second set of capture probes from the array. In some embodiments, hybridizing the second analyte to the second capture domain is performed after releasing the second set of capture probes from the array.

In some embodiments, releasing the first set of capture probes from the array is performed after the ligation product hybridizes to the first capture domain. In some embodiments, hybridizing the ligation product to the first capture domain is performed after releasing the first set of capture probes from the array. In some embodiments, the method includes hybridizing a ligation product from a second analyte or a ligation product from a second biological sample to the second capture domain.

In some embodiments, hybridizing the ligation product from the second analyte or the ligation product from a second biological sample is performed before releasing the second set of capture probes from the array. In some embodiments, hybridizing the ligation product from the second analyte or the ligation product from a second biological sample to the second capture domain is performed after releasing the second set of capture probes from the array.

In some embodiments, the method includes hybridizing one or more additional ligation products from one or more additional analytes to a capture probe in the one or more additional sets of capture probes, and releasing the one or more additional sets of capture probes from the array.

In some embodiments, the method includes releasing the ligation product from the analyte. In some embodiments, releasing includes the use of an RNase (e.g., any suitable RNase known in the art, such as RNAse H).

In some embodiments, the method includes extending the first capture probe using the analyte as a template (e.g., an extension template). In some embodiments, the method includes extending the first capture probe using the ligation product as a template (e.g., an extension template) and/or extending the ligation product using the first capture probe as a template.

In some embodiments, when the biological sample is on a different substrate than the array including two or more sets of capture probes, at least a portion of the biological sample is aligned with at least a portion of the array.

In some embodiments, the sequence of the first spatial barcode and the second spatial barcode are unique to a distinct position on the array. In some embodiments, the first spatial barcode and the second spatial barcode include the same sequence, e.g., corresponding to the same position on the array. In some embodiments, the first spatial barcode and the second spatial barcode include different sequences, e.g., corresponding to different positions on the array.

In some embodiments, the first capture domain and the second capture domain include different sequences. For example, the first capture domain sequence can be specific to a gene or a family of genes. In some embodiments, the first capture domain and the second capture domain include the same sequence. For example, the first capture domain and the second capture domain can both have a poly(T) or poly(U) capture sequence.

In some embodiments, coupling includes ligating the first probe and the second probe via a ligase. In some embodiments, the ligase is one or more of a T4 RNA ligase (Rnl2), a PBCV-1 DNA ligase, a Chorella virus DNA ligase, a single-stranded DNA ligase, a T4 DNA ligase, and combinations thereof.

In some embodiments, the first probe and/or the second probe includes a primer sequence.

In some embodiments, hybridizing the first probe and the second probe to the analyte includes contacting the biological sample with about 5,000 or more probe pairs collectively including the first probe and the second probe. In some embodiments, the first probe and/or the second probe is a DNA probe.

In some embodiments, the method includes releasing the ligation product from the analyte, where releasing includes contacting the biological sample (e.g., the ligation product therein) with an endoribonuclease. In some embodiments, the endoribonuclease is an RNase enzyme. In some embodiments, the RNase is RNase H.

In some embodiments, the first probe and the second probe are substantially complementary to adjacent sequences of the analyte (e.g., the first probe and second probe abut one another when hybridized to the analyte). In some embodiments, the first probe and the second probe hybridize to sequences that are not adjacent to each other on the analyte. For example, when hybridized to the analyte, an end of the first and an end of the second probe can be 1, 2, 3, 4, 5, 6, 7, 8, 9, 10, 15, 20 or more nucleotides apart. In some embodiments, the first probe is extended, e.g., with a DNA polymerase, thereby (i) filling in a gap between the first probe and the second probe and (ii) generating an extended first probe. In such embodiments, the extended first probe is ligated to the second probe.

In some embodiments, the analyte is a nucleic acid. In some embodiments, the nucleic acid is an RNA molecule. In some embodiments, the RNA molecule is mRNA. In some embodiments, the nucleic acid is a DNA molecule. In some embodiments, the DNA molecule is genomic DNA.

In some embodiments, the method includes contacting the biological sample with one or more analyte capture agents, where an analyte capture agent includes: (i) an analyte binding moiety and (ii) an oligonucleotide including an analyte binding moiety barcode and an analyte capture sequence.

In some embodiments, the analyte binding moiety barcode identifies the analyte (e.g., the barcode is a unique sequence that identifies the analyte bound by the analyte binding moiety). In some embodiments, the analyte is a protein.

In some embodiments, the analyte capture sequence hybridizes to the second capture domain of the second capture probe.

In some embodiments, the analyte binding moiety is an antibody or an antigen binding fragment thereof.

In some embodiments, the method includes i) extending the second capture probe using the oligonucleotide as a template, thereby generating an extended capture probe including the second spatial barcode, the analyte binding moiety barcode or a complement thereof, optionally, a unique molecular identifier (UMI) of the second capture probe, and optionally an analyte capture sequence or a complement thereof; and/or ii) extending the oligonucleotide using the second capture probe as a template, thereby generating an extended oligonucleotide.

In some embodiments, the method includes amplifying all or a portion of the extended capture probe, or a complement thereof.

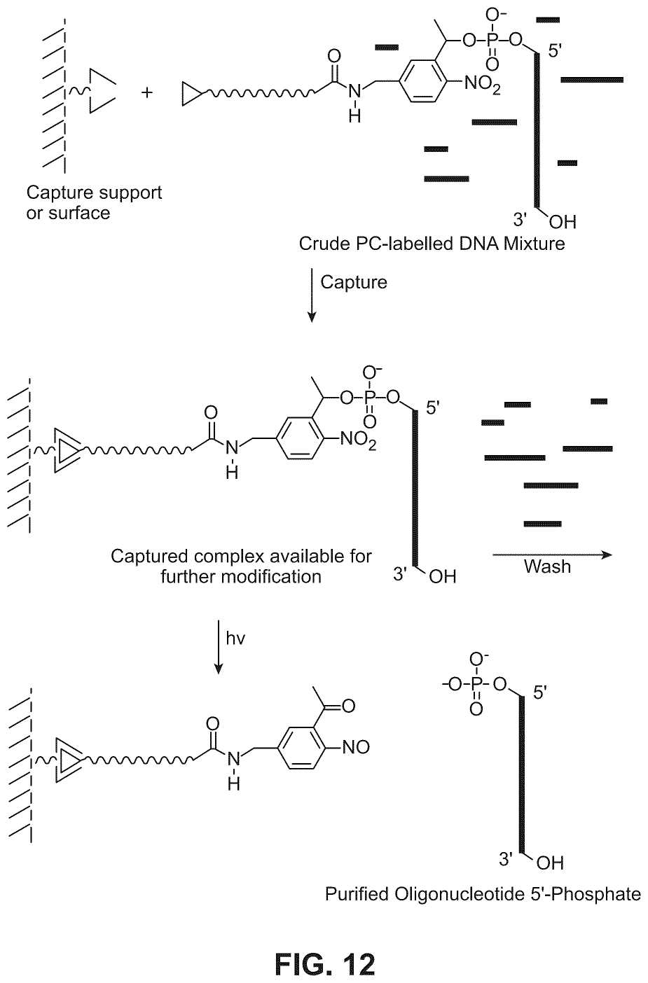

FIG. 12 shows an exemplary photocleavable site containing a photocleavable oligonucleotide that can be used in the methods described herein. More specifically, FIG. 12 shows 5′-PC-amino modifier CE phosphoramidite which can be incorporated into the capture probe on an array.

In some embodiments, the first photocleavable cleavage site and the second photocleavable cleavage site comprise one or more nucleosides comprising a photoreactive group.

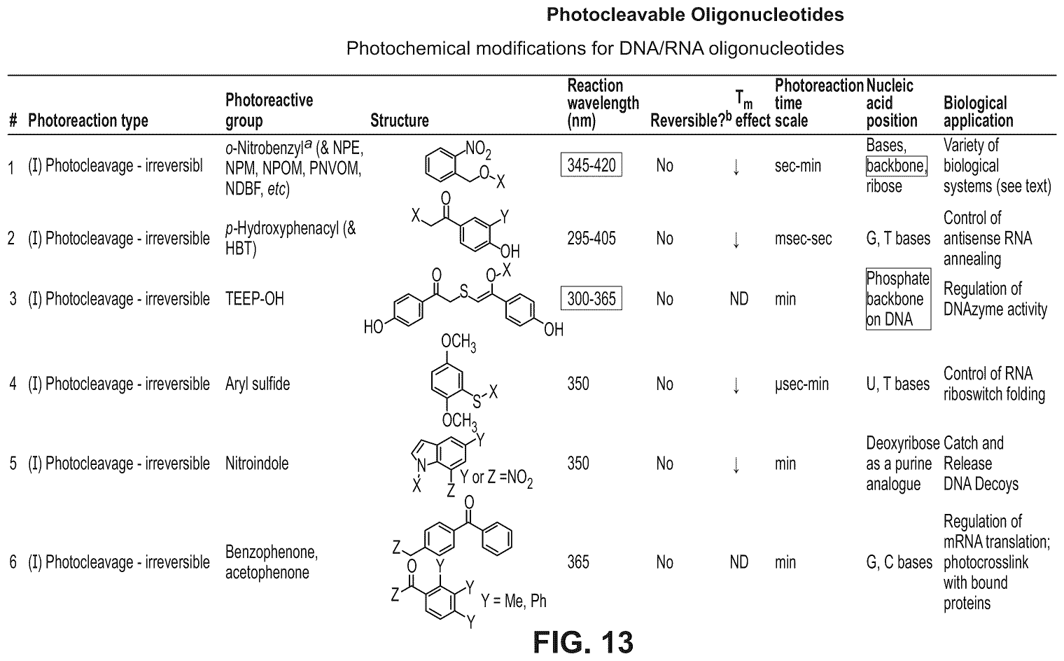

In some embodiments, the photoreactive group is selected from o-Nitrobenzyl (o-NB) derivatives, p-Hydroxyphenacyl, TEEP-OH, Aryl sulfide, and nitroindole. FIG. 13 provides exemplary photoreactive groups and their structures.

### (iii) Sandwich Processes

In some embodiments, analytes or analyte derived products from the biological sample are released from the biological sample and captured by any of the methods described herein. The resulting products (e.g., ligation product, probes, extended probes, etc.) can be captured by capture probes immobilized on a substrate or by released capture probes. In some embodiments, the biological sample is mounted on a first substrate and the substrate comprising the array of capture probes is a second substrate. In some embodiments, the alignment of the first substrate and the second substrate is facilitated by a sandwiching process. Accordingly, described herein are methods, compositions, and kits for sandwiching together the first substrate as described herein with a second substrate having an array with capture probes.

In some embodiments, the sandwiching process may be facilitated by a device, sample holder, sample handling apparatus, or system described in, e.g., US. Patent Application Pub. No. 20210189475, WO 2021/252747, or WO 2022/061152.

In some embodiments, the first and second substrates are placed in a substrate holder (e.g., an array alignment device) configured to align the biological sample and the array. In some embodiments, the device comprises a sample holder. In some embodiments, the sample holder includes a first member and a second member that receive a first substrate and a second substrate, respectively. The device can include an alignment mechanism that is connected to at least one of the members and aligns the first and second members. Thus, the devices of the disclosure can advantageously align the first substrate and the second substrate and any samples, barcoded probes, or permeabilization reagents that may be on the surface of the first and second substrates.

In some embodiments, the sandwiching process comprises: mounting the first substrate on a first member of a support device, the first member configured to retain the first substrate; mounting the second substrate on a second member of the support device, the second member configured to retain the second substrate, applying a reagent medium to the first substrate and/or the second substrate, the reagent medium comprising a permeabilization agent, operating an alignment mechanism of the support device to move the first member and/or the second member such that a portion of the biological sample is aligned (e.g., vertically aligned) with a portion of the array of capture probes and within a threshold distance of the array of capture probes, and such that the portion of the biological sample and the capture probe contact the reagent medium, wherein the permeabilization agent releases the analytes or analyte derived products from the biological sample.

In some embodiments of a sample holder, the sample holder can include a first member including a first retaining mechanism configured to retain a first substrate comprising a sample. The first retaining mechanism can be configured to retain the first substrate disposed in a first plane. The sample holder can further include a second member including a second retaining mechanism configured to retain a second substrate disposed in a second plane. The sample holder can further includes an alignment mechanism connected to one or both of the first member and the second member. The alignment mechanism can be configured to align the first and second members along the first plane and/or the second plane such that the sample contacts at least a portion of the reagent medium when the first and second members are aligned and within a threshold distance along an axis orthogonal to the second plane. The alignment mechanism may be configured to move the second member along the axis orthogonal to the second plane and/or move the first member along an axis orthogonal to the first plane.

In some embodiments, the alignment mechanism includes a linear actuator. In some embodiments, the alignment mechanism includes one or more of a moving plate, a bushing, a shoulder screw, a motor bracket, and a linear actuator. The moving plate may be coupled to the first member or the second member. The alignment mechanism may, in some cases, include a first moving plate coupled to the first member and a second moving plate coupled to the second member. In some embodiments, the linear actuator is configured to move the second member along an axis orthogonal to the plane of the first member and/or the second member. For example, the moving plate may be coupled to the second member and adjust the separation distance along a z axis (e.g., orthogonal to the second substrate) by moving the moving plate up in a superior direction toward the first substrate. In some embodiments, the linear actuator is configured to move the first member along an axis orthogonal to the plane of the first member and/or the second member. The movement of the moving plate may be accomplished by the linear actuator configured to move the first member and/or the second member at a velocity. The velocity may be controlled by a controller communicatively coupled to the linear actuator. In some embodiments, the linear actuator is configured to move the first member, the second member, or both the first member and the second member at a velocity of at least 0.1 mm/sec (e.g., at least 0.1 mm/sec to 2 mm/sec). In some aspects, the velocity may be selected to reduce or minimize bubble generation or trapping within the reagent medium. In some embodiments, the linear actuator is configured to move the first member, the second member, or both the first member and the second member with an amount of force of at least 0.1 lbs (e.g., between 0.1-4.0 pounds of force).

In some aspects, the velocity of the moving plate (e.g., closing the sandwich) may affect bubble generation or trapping within the reagent medium. It may be advantageous to minimize bubble generation or trapping within the reagent medium during the “sandwiching” process, as bubbles can interfere with the migration of analytes or analyte derived products through the reagent medium to the array. In some embodiments, the closing speed is selected to minimize bubble generation or trapping within the reagent medium. In some embodiments, the closing speed is selected to reduce the time it takes the flow front of the reagent medium from an initial point of contact with the first and second substrate to sweep across the sandwich area (also referred to herein as “closing time”). In some embodiments, the closing speed is selected to reduce the closing time to less than about 1100 milliseconds (ms). In some embodiments, the closing speed is selected to reduce the closing time to less than about 1000 ms. In some embodiments, the closing speed is selected to reduce the closing time to less than about 900 ms. In some embodiments, the closing speed is selected to reduce the closing time to less than about 750 ms. In some embodiments, the closing speed is selected to reduce the closing time to less than about 600 ms. In some embodiments, the closing speed is selected to reduce the closing time to about 550 ms or less. In some embodiments, the closing speed is selected to reduce the closing time to about 370 ms or less. In some embodiments, the closing speed is selected to reduce the closing time to about 200 ms or less. In some embodiments, the closing speed is selected to reduce the closing time to about 150 ms or less.

Further details on support devices, sample holders, sample handling apparatuses, or systems for implementing a sandwiching process are described in, e.g., PCT Publ. No. WO 2021/0189475 and WO 2022/061152, each of which are incorporated by reference in their entireties.

Analytes or analyte derived products within a biological sample may be released through disruption (e.g., permeabilization, digestion, etc.) of the biological sample or may be released without disruption. Various methods of permeabilizing (e.g., any of the permeabilization reagents and/or conditions described herein) a biological sample are described herein, including for example including the use of various detergents, buffers, proteases, and/or nucleases for different periods of time and at various temperatures. Additionally, various methods of delivering fluids (e.g., a buffer, a permeabilization solution) to a biological sample are described herein including the use of a substrate holder (e.g., for sandwich assembly, sandwich configuration, as described herein)

Provided herein are methods for delivering a fluid to a biological sample disposed on an area of a first substrate and an array disposed on a second substrate.

In some embodiments, the sandwich configuration described herein between a first substrate comprising a biological sample and a second substrate comprising a spatially barcoded array may include a reagent medium comprising NaCl, ethylene carbonate, and/or glycerol to fill a gap. It may be desirable that the reagent medium be free from air bubbles between the slides to facilitate transfer of target molecules with spatial information. Additionally, air bubbles present between the slides may obscure at least a portion of an image capture of a desired region of interest. Accordingly, it may be desirable to ensure or encourage suppression and/or elimination of air bubbles between the two substrates during a permeabilization step.

In some aspects, it may be possible to reduce or eliminate bubble formation between the slides using a variety of filling methods and/or closing methods.

In some embodiments, the contacting comprises bringing the two substrates into proximity such that the sample on the first substrate is aligned with the barcode array of capture probes on the second substrate.

In some embodiments, the drop includes permeabilization reagents (e.g., any of the permeabilization reagents described herein). In some embodiments, the rate of permeabilization of the biological sample is modulated by delivering the permeabilization reagents (e.g., a fluid containing permeabilization reagents) at various temperatures.

Robust fluidics in the sandwich making described herein may preserve spatial information by reducing or preventing deflection of molecules as they move from the tissue slide to the capture slide.

Further details on angled closure workflows, and devices and systems for implementing an angled closure workflow, are described in WO 2021/252747 and WO 2022/061152, which are hereby incorporated by reference in their entireties. Additional configurations for reducing or eliminating bubble formation, and/or for reducing unwanted fluid flow, are described in WO 2021/252747, which is hereby incorporated by reference in its entirety.

Suitable permeabilization agents include, but are not limited to, organic solvents (e.g., acetone, ethanol, and methanol), cross-linking agents (e.g., paraformaldehyde), detergents (e.g., saponin, Triton X-100™, Tween-20™, or sodium dodecyl sulfate (SDS)), and enzymes (e.g., trypsin, proteases (e.g., proteinase K). In some embodiments, the detergent is an anionic detergent (e.g., SDS or N-lauroylsarcosine sodium salt solution). Exemplary permeabilization reagents are described in in PCT Patent Application Publication No. WO 2020/123320, which is incorporated by reference in its entirety.

Lysis solutions can include ionic surfactants such as, for example, sarkosyl and sodium dodecyl sulfate (SDS). More generally, chemical lysis agents can include, without limitation, organic solvents, chelating agents, detergents, surfactants, and chaotropic agents. Exemplary lysis reagents are described in PCT Patent Application Publication No. WO 2020/123320, which is incorporated by reference in its entirety.

Exemplary proteases include, e.g., pepsin, trypsin, pepsin, elastase, and proteinase K. Exemplary proteases are described in PCT Patent Application Publication No. WO 2020/123320, which is incorporated by reference in its entirety. Exemplary detergents include sodium dodecyl sulfate (SDS), sarkosyl, saponin, Triton X-100™, and Tween-20™. Exemplary detergents are described in PCT Patent Application Publication No. WO 2020/123320, which is incorporated by reference in its entirety.

In some embodiments, the reagent medium comprising NaCl, ethylene carbonate, and/or glycerol also comprises a nuclease. In some embodiments, the nuclease comprises am RNase. In some embodiments, the RNase is selected from RNase A, RNase C, RNase H, and RNase I. In some embodiments, the reagent medium comprises one or more of sodium dodecyl sulfate (SDS), proteinase K, pepsin, N-lauroylsarcosine, RNAse, and a sodium salt thereof.

The sample holder is compatible with a variety of different schemes for contacting the aligned portions of the biological sample and array with the reagent medium to promote capture of the analytes or analyte derived products. In some embodiments, the reagent medium is deposited directly on the second substrate (e.g., forming a reagent medium that includes the permeabilization reagent and the feature array), and/or directly on the first substrate. In some embodiments, the reagent medium is deposited on the first and/or second substrate, and then the first and second substrates aligned in the sandwich configuration such that the reagent medium contacts the aligned portions of the biological sample and array. In some embodiments, the reagent medium is introduced into the gap while the first and second substrates are aligned in the sandwich configuration.

In certain embodiments a dried permeabilization reagent is applied or formed as a layer on the first substrate or the second substrate or both prior to contacting the sample and the feature array. For example, a reagent can be deposited in solution on the first substrate or the second substrate or both and then dried. Drying methods include, but are not limited to spin coating a thin solution of the reagent and then evaporating a solvent included in the reagent or the reagent itself. Alternatively, in other embodiments, the reagent can be applied in dried form directly onto the first substrate or the second substrate or both. In some embodiments, the coating process can be done in advance of the analytical workflow and the first substrate and the second substrate can be stored pre-coated. Alternatively, the coating process can be done as part of the analytical workflow. In some embodiments, the reagent is a permeabilization reagent. In some embodiments, the reagent is a permeabilization enzyme, a buffer, a detergent, or any combination thereof. In some embodiments, the permeabilization enzyme is pepsin. In some embodiments, the reagent is a dried reagent (e.g., a reagent free from moisture or liquid). In some instances, the substrate that includes the sample (e.g., a histological tissue section) is hydrated. The sample can be hydrated by contacting the sample with a reagent medium, e.g., a buffer that does not include a permeabilization reagent. In some embodiments, the hydration is performed while the first and second substrates are aligned in a sandwich configuration.

In some embodiments, following initial contact between sample and a permeabilization agent, the permeabilization agent can be removed from contact with sample (e.g., by opening sample holder) before complete permeabilization of sample. For example, in some embodiments, only a portion of sample is permeabilized, and only a portion of the analytes or analyte derived products in the sample may be captured by the feature array. In some instances, the reduced amount of analytes or analyte derived products captured and available for detection can be offset by the reduction in lateral diffusion that results from incomplete permeabilization of sample. In general, the spatial resolution of the assay is determined by the extent of analytes or analyte derived products diffusion in the transverse direction (i.e., orthogonal to the normal direction to the surface of sample). The larger the distance between the sample on the first substrate and the feature array on the second substrate, the greater the extent of diffusion in the transverse direction, and the concomitant loss of resolution. Analytes or analyte derived products liberated from a portion of the sample closest to the feature array have a shorter diffusion path, and therefore do not diffuse as far laterally as analytes or analyte derived products from portions of the sample farthest from the feature array. As a result, in some instances, incomplete permeabilization of the sample (by reducing the contact interval between the permeabilization agent and the sample) can be used to maintain adequate spatial resolution in the assay.

In some instances, the device is configured to control a temperature of the first and second substrates. In some embodiments, the temperature of the first and second members is lowered to a first temperature that is below room temperature (e.g., 25 degrees Celsius) (e.g., 20 degrees Celsius or lower, 15 degrees Celsius or lower, 10 degrees Celsius or lower, 5 degrees Celsius or lower, 4 degrees Celsius or lower, 3 degrees Celsius or lower, 2 degrees Celsius or lower, 1 degree Celsius or lower, 0 degrees Celsius or lower, −1 degrees Celsius or lower, −5 degrees Celsius or lower). In some embodiments, the device includes a temperature control system (e.g., heating and cooling conducting coils) to control the temperature of the sample holder. Alternatively, in other embodiments, the temperature of the sample holder is controlled externally (e.g., via refrigeration or a hotplate). In a first step, the second member, set to or at the first temperature, contacts the first substrate, and the first member, set to or at the first temperature, contacts the second substrate, thereby lowering the temperature of the first substrate and the second substrate to a second temperature. In some embodiments, the second temperature is equivalent to the first temperature. In some embodiments, the first temperature is lower than room temperature (e.g., 25 degrees Celsius). In some embodiments, the second temperature ranges from about-10 degrees Celsius to about 4 degrees Celsius. In some embodiments, the second temperature is below room temperature (e.g., 25 degrees Celsius) (e.g., 20 degrees Celsius or lower, 15 degrees Celsius or lower, 10 degrees Celsius or lower, 5 degrees Celsius or lower, 4 degrees Celsius or lower, 3 degrees Celsius or lower, 2 degrees Celsius or lower, 1 degree Celsius or lower, 0 degrees Celsius or lower, −1 degrees Celsius or lower, −5 degrees Celsius or lower).

In an exemplary embodiment, the second substrate is contacted with the permeabilization reagent. In some embodiments, the permeabilization reagent is dried. In some embodiments, the permeabilization reagent is a gel or a liquid. Also in the exemplary embodiment, the biological sample is contacted with buffer. Both the first and second substrates are placed at lower temperature to slow down diffusion and permeabilization efficiency. Alternatively, in some embodiments, the sample can be contacted directly with a liquid permeabilization reagent without inducing an unwanted initiation of permeabilization due to the substrates being at the second temperature. In some embodiments, the low temperature slows down or prevents the initiation of permeabilization. In a second step, keeping the sample holder and substrates at a cold temperature (e.g., at the first or second temperatures) continues to slow down or prevent the permeabilization of the sample. In a third step, the sample holder (and consequently the first and second substrates) is heated up to initiate permeabilization. In some embodiments, the sample holder is heated up to a third temperature. In some embodiments, the third temperature is above room temperature (e.g., 25 degrees Celsius) (e.g., 30 degrees Celsius or higher, 35 degrees Celsius or higher, 40 degrees Celsius or higher, 50 degrees Celsius or higher, 60 degrees Celsius or higher). In some embodiments, analytes or analyte derived products that are released from the permeabilized tissue of the sample diffuse to the surface of the second substrate and are captured on the array (e.g., barcoded probes) of the second substrate. In a fourth step, the first substrate and the second substrate are separated (e.g., pulled apart) and temperature control is stopped.

In some embodiments, where either the first substrate or substrate second (or both) includes wells, a permeabilization solution can be introduced into some or all of the wells, and then the sample and the feature array can be contacted by closing the sample holder to permeabilize the sample. In certain embodiments, a permeabilization solution can be soaked into a hydrogel film that is applied directly to the sample, and/or soaked into features (e.g., beads) of the array. When the first and second substrates are aligned in the sandwich configuration, the permeabilization solution promotes migration of products derived from analytes or analyte derived products from the sample to the array.

In certain embodiments, different permeabilization agents or different concentrations of permeabilization agents can be infused into array features (e.g., beads) or into a hydrogel layer as described above. By locally varying the nature of the permeabilization reagent(s), the process of analytes or analyte derived products capture from the sample can be spatially adjusted.

In some instances, migration of the analytes or analyte derived products from the biological sample to the second substrate is passive (e.g., via diffusion). Alternatively, in certain embodiments, migration of the analytes or analyte derived products from the biological sample is performed actively (e.g., electrophoretic, by applying an electric field to promote migration). In some instances, first and second substrates can include a conductive epoxy. Electrical wires from a power supply can connect to the conductive epoxy, thereby allowing a user to apply a current and generate an electric field between the first and second substrates. In some embodiments, electrophoretic migration results in higher capture efficiency of analytes or analyte derived products and better spatial fidelity of captured analytes or analyte derived products (e.g., on a feature array) than random diffusion onto matched substrates without the application of an electric field (e.g., via manual alignment of the two substrates). Exemplary methods of electrophoretic migration are described in WO 2020/176788, including at FIGS. 13-15, 24A-24B, and 25A-25C, which is hereby incorporated by reference in its entirety.

Loss of spatial resolution can occur when analytes or analyte derived products migrate from the sample to the feature array and a component of diffusive migration occurs in the transverse (e.g., lateral) direction, approximately parallel to the surface of the first substrate on which the sample is mounted. To address this loss of resolution, in some embodiments, a permeabilization agent deposited on or infused into a material with anisotropic diffusion can be applied to the sample or to the feature array. The first and second substrates are aligned by the sample holder and brought into contact. A permeabilization layer that includes a permeabilization solution infused into an anisotropic material is positioned on the second substrate.

In some embodiments, the feature array can be constructed atop a hydrogel layer infused with a permeabilization agent. The hydrogel layer can be mounted on the second substrate, or alternatively, the hydrogel layer itself may function as the second substrate. When the first and second substrates are aligned, the permeabilization agent diffuses out of the hydrogel layer and through or around the feature array to reach the sample. Analytes or analyte derived products from the sample migrate to the feature array. Direct contact between the feature array and the sample helps to reduce lateral diffusion of the analytes or analyte derived products, mitigating spatial resolution loss that would occur if the diffusive path of the analytes or analyte derived products was longer.

Spatial analysis workflows can include a sandwiching process described herein. In some embodiments, the workflow includes provision of the first substrate comprising the biological sample. In some embodiments, the workflow includes, mounting the biological sample onto the first substrate. In some embodiments wherein the biological sample is a tissue sample, the workflow include sectioning of the tissue sample (e.g., cryostat sectioning). In some embodiments, the workflow includes a fixation step. In some instances, the fixation step can include fixation with methanol. In some instances, the fixation step includes formalin (e.g., 2% formalin).

In some embodiments, the biological sample on the first substrate is stained using any of the methods described herein. In some instances, the biological sample is imaged, capturing the stain pattern created during the stain step. In some instances, the biological sample then is destained prior to the sandwiching process.

In some instances, the methods include imaging the biological sample. In some instances, imaging occurs prior to sandwich assembly. In some instances, imaging occurs while the sandwich configuration is assembled. In some instances, imaging occurs during permeabilization of the biological sample. In some instances, image are captured using high resolution techniques (e.g., having 300 dots per square inch (dpi) or greater). For example, images can be captured using brightfield imaging (e.g., in the setting of hematoxylin or H&E stain), or using fluorescence microscopy to detect adhered labels. In some instances, high resolution images are captured temporally using e.g., confocal microscopy. In some instances, a low resolution image is captured. A low resolution image (e.g., images that are about 72 dpi and normally have an RGB color setting) can be captured at any point of the workflow, including but not limited to staining, destaining, permeabilization, sandwich assembly, and migration of the analytes or analyte derived products. In some instances, a low resolution image is taken during permeabilization of the biological sample.

In some instances, the biological samples can be destained. In some instances, destaining occurs prior to permeabilization of the biological sample. By way of example only, H&E staining can be destained by washing the sample in HCl. In some instances, the hematoxylin of the H&E stain is destained by washing the sample in HCl. In some embodiments, destaining can include 1, 2, 3, or more washes in HCl. In some embodiments, destaining can include adding HCl to a downstream solution (e.g., permeabilization solution).

Between any of the methods disclosed herein, the methods can include a wash step (e.g., with SSC (e.g., 0.1×SSC)). Wash steps can be performed once or multiple times (e.g., 1×, 2×, 3×, between steps disclosed herein). In some instances, wash steps are performed for about 10 seconds, about 15 seconds, about 20 seconds, about 30 seconds, or about a minute. In some instances, three washes occur for 20 seconds each. In some instances, the wash step occurs before staining the sample, after destaining the sample, before permeabilization the sample, after permeabilization the sample, or any combination thereof.

In some instances, after the sandwiching process the first substrate and the second substrate are separated (e.g., such that they are no longer aligned in a sandwich configuration, also referred to herein as opening the sandwich). In some embodiments, subsequent analysis (e.g., cDNA synthesis, library preparation, and sequences) can be performed on the captured analytes or analyte derived products after the first substrate and the second substrate are separated.

In some embodiments, the process of transferring the products derived from analytes or analyte derived products from the first substrate to the second substrate is referred to interchangeably herein as a “sandwich process,” “sandwiching process,” or “sandwiching”. The sandwich process is further described in PCT Patent Application Publication No. WO 2020/123320, PCT/US2021/036788, and PCT/US2021/050931, which are incorporated by reference in their entireties.

### (iv) Library Preparation

In some embodiments, the analytes or complements thereof and other analyte derived products (e.g., a ligation product, an oligonucleotide including an analyte binding moiety barcode), and/or amplicons of such products, can be prepared for downstream applications, such as generation of a sequencing library and next-generation sequencing. Generating sequencing libraries are known in the art. For example, the analyte or complements thereof can be purified and collected for downstream amplification steps. The amplification products can be amplified using PCR, where primer binding sites flank the spatial barcode and target nucleic acid, or a complement thereof, generating a library associated with a particular spatial barcode. In some embodiments, the library preparation can be quantitated and/or quality controlled to verify the success of the library preparation steps. The library amplicons are sequenced and analyzed to decode spatial information and the analyte or analyte-derived product (e.g., a ligation product, an oligonucleotide including an analyte binding moiety barcode).

Alternatively or additionally, the amplicons can then be enzymatically fragmented and/or size-selected in order to provide for desired amplicon size. In some embodiments, when utilizing an Illumina® library preparation methodology, for example, P5 and P7, sequences can be added to the amplicons thereby allowing for capture of the library preparation on a sequencing flowcell (e.g., on Illumina sequencing instruments). Additionally, i7 and i5 can index sequences be added as sample indexes if multiple libraries are to be pooled and sequenced together. Further, Read 1 and Read 2 sequences can be added to the library for sequencing purposes. The aforementioned sequences can be added to a library preparation sample, for example, via End Repair, A-tailing, Adaptor Ligation, and/or PCR. The cDNA fragments can then be sequenced using, for example, paired-end sequencing using TruSeq Read 1 and TruSeq Read 2 as sequencing primer sites, although other methods are known in the art.

### (v) Kits

The present disclosure also features kits including reusable arrays. Thus, provided herein are kits including: an array including a first set of capture probes and a second set of capture probes, where a first capture probe of the first set of capture probes includes: (i) a first spatial barcode; (ii) a first capture domain; and (iii) a first restriction endonuclease cleavage site; and where a second capture probe of the second set of capture probes includes: (i) a second spatial barcode; (ii) a second capture domain; and (iii) a second restriction endonuclease cleavage site; where the sequence of the first restriction endonuclease cleavage site of the first capture probe differs from the sequence of the second restriction endonuclease cleavage site of the second capture probe.

Also provided herein are kits including: an array including a first set of capture probes and a second set of capture probes, where a first capture probe of the first set of capture probes includes: (i) a first spatial barcode; (ii) a first capture domain; and (iii) a first photocleavable cleavage site; and where a second capture probe of the second set of capture probes includes: (i) a second spatial barcode; (ii) a second capture domain; and (iii) a second photocleavable cleavage site; where the first photocleavable cleavage site of the first capture probe differs from the second photocleavable cleavage site of the second capture probe.

In some embodiments, the kit includes a plurality of first probes and a plurality of second probes, where a first probe of the plurality of first probes and a second probe of the plurality of second probes each include a sequence that is substantially complementary to sequences of an analyte, and where the second probe includes a capture probe binding domain. In some embodiments, the kit includes 5,000, 10,000, 15,000, 18,000 or more probe pairs.

In some embodiments, the kit includes a plurality of analyte capture agents, where an analyte capture agent includes: (i) an analyte binding moiety; (ii) an oligonucleotide including an analyte binding moiety barcode and an analyte capture sequence.

In some embodiments, the kit includes one or more permeabilization reagents selected from protease, pepsin, proteinase K, and collagenase. In some embodiments, the kit includes a detergent.

The kits described herein can also include one or more enzymes. In some embodiments, the kit includes one or more of a ligase (e.g., any of the ligases described herein), a DNA polymerase, a reverse transcriptase, an RNase (e.g., RNase H), and combinations thereof. In some embodiments, the kit includes one or more crosslinking agents. In some embodiments, the kit includes one or more decrosslinking agents. In some embodiments, the kit includes instructions for performing any of the methods described herein.

### (vi) Compositions

In addition to the methods and kits described herein, the present disclosure features compositions including reusable arrays. Thus, provided herein are compositions, including: an array including a first set of capture probes and a second set of capture probes, where a first capture probe of the first set of capture probes includes: (i) a first spatial barcode; (ii) a first capture domain; and (iii) a first restriction endonuclease cleavage site; and where a second capture probe of the second set of capture probes includes: (i) a second spatial barcode; (ii) a second capture domain; (iii) a second restriction endonuclease cleavage site; where the sequence of the first restriction endonuclease cleavage site of the first capture probe differs from the sequence of the second restriction endonuclease cleavage site of the second capture probe.

Also provided herein are compositions, including: an array including a first set of capture probes and a second set of capture probes, where a first capture probe of the first set of capture probes includes: (i) a first spatial barcode; (ii) a first capture domain; and (iii) a first photocleavable cleavage site; and where a second capture probe of the second set of capture probes includes: (i) a second spatial barcode; (ii) a second capture domain; and (iii) a second photocleavable cleavage site; where the first photocleavable cleavage site of the first capture probe differs from the second photocleavable cleavage site of the second capture probe.

In some embodiments, the composition includes the first capture probe released from the array and hybridized to an analyte in the biological sample. In some embodiments, the composition includes an extended first capture probe where the analyte was used as a template (e.g., an extension template).

In some embodiments, the composition includes the analyte hybridized to the first capture probe on the array. In some embodiments, the composition includes an extended first capture probe on the array, where the analyte was used as a template (e.g., an extension template).

In some embodiments, the composition includes a second capture probe hybridized to a second analyte or an analyte from a second biological sample. In some embodiments, the second capture probe was released prior to hybridizing to the second analyte or the analyte from the second biological sample. In some embodiments, the second capture probe was released after hybridizing to the second analyte or the analyte from the second biological sample.

In some embodiments, the composition includes one or more enzymes. In some embodiments, the composition includes an RNase (e.g., any of the RNases described herein). In some embodiments, the composition includes a polymerase (e.g., a DNA polymerase). In some embodiments, the composition includes a reverse transcriptase. The kit can also include additional reagents. For example, the composition can include one or more permeabilization reagents (e.g., any of the permeabilization reagents described herein), one or more a cross-linking agents, one or more decrosslinking agents, and combinations thereof.

In some embodiments, the composition includes a plurality of first probes and a plurality of second probes, where a first probe of the plurality of first probes and a second probe of the plurality of second probes each include a sequence that is substantially complementary to sequences of the analyte, and where the second probe includes a capture probe binding domain.

In some embodiments, the first probe and the second probe are coupled to form a ligation product with the use of a ligase.

In some embodiments, the composition includes a first capture probe released from the array and hybridized to the ligation product in a biological sample. In some embodiments, the composition includes an extended first capture probe using the ligation product as a template (e.g., an extension template).

In some embodiments, the composition includes a hybridized ligation product to the first capture probe on the array. In some embodiments, the composition includes an extended first capture probe using the ligation product as a template (e.g., an extension template) on the array.

In some embodiments, the composition includes a plurality of analyte capture agents, where an analyte capture agent includes: (i) an analyte binding moiety and (ii) an oligonucleotide including an analyte binding moiety barcode and an analyte capture sequence.

In some embodiments, the composition includes a second capture probe released from the array and hybridized to the analyte capture sequence of the analyte capture agent in a biological sample. In some embodiments, the composition includes an extended second capture probe where the analyte capture sequence was used as a template.

In some embodiments, the array includes one or more additional sets of capture probes.

### EXAMPLES

### Example 1. Reusable Spatial Arrays for Capturing and Determining the Spatial Location of an Analyte

Provided herein are reusable spatial arrays for capturing and determining the spatial location of an analyte based on the use of removable spatially barcoded capture probes. The reusable array has different sets of capture probes where each capture probe set has a unique release mechanism (e.g., any of the release mechanisms described herein).

In some examples, analytes or analyte derived products (e.g., ligation products, oligonucleotides from analyte capture agents) from a biological sample hybridize to the capture probes on the array. In some embodiments, the capture probes contain capture domains specific to a desired analyte, e.g., a gene specific sequence, or a sequence specific to a class of analytes. In some embodiments, a first set of capture probes on the array is used for the hybridization while one or more additional sets of capture probes on the array are blocked to prevent or reduce hybridization to the analytes or analyte derived products. The capture probes which are hybridized to the analytes or analyte derived products are extended (e.g., by a polymerase) to generate extended capture probes including a complement of all or a portion of the analyte or analyte derived product. Additionally, and/or alternatively, the analyte derived products can be extended e.g., by a polymerase using the capture probe as templates. Upon completion of extension, the extended capture probes are released from the array (e.g., enzymatically or by photocleavage), captured, and subject to downstream library preparation, while the remaining capture probes on the array are subsequently used to capture other analytes or analyte derived molecules e.g., from the same biological sample or a different biological sample.

In some examples, the capture probes are released from the array and hybridize to the analyte (e.g., nucleic acid) or analyte derived products off the array (e.g., in or on a biological sample). In some embodiments, the released capture probes contain capture domains specific to a desired analyte, e.g., a gene specific sequence, or a sequence specific to a class of analytes. In some embodiments, the released capture probes comprise a first set of capture probes from the array, while one or more additional sets of capture probes remain attached to the array. The released capture probes which are hybridized to the analytes or analyte derived products are extended (e.g., by a polymerase) to generate extended capture probes including a complement of all or a portion of the analyte or analyte derived product. Additionally, or alternatively, the analyte derived products can be extended e.g., by a polymerase using the capture probe as templates. The extended capture probes or extended analyte derived products (e.g., extended ligation products) are captured, and subject to downstream library preparation, while the remaining capture probes on the array are subsequently used to capture other analytes or analyte derived molecules e.g., from the same or different biological sample.

Analyte derived products include products such as a ligation products and/or oligonucleotides that include an analyte capture sequence and an analyte binding moiety barcode associated with an analyte capture agent.

Analytes or analyte derived products can be captured sequentially from the same biological sample (e.g., a second analyte, a third analyte, or more). Additionally, and/or alternatively, analytes or analyte derived products can be captured from additional biological samples (e.g., a second biological sample, a third biological sample, or more).

The reusable arrays described herein lower the cost per reaction for the use of spatial arrays. Another benefit of using the arrays described herein is that any potential steric hindrance is reduced for the next round of use after removal of a first set of capture probes.

1.-125. (canceled)

126. A method for determining a spatial location of an analyte in a biological sample, the method comprising:
(a) disposing the biological sample onto a first substrate;
(b) providing a second substrate comprising an array, wherein the array comprises a first set of capture probes and a second set of capture probes,
wherein a first capture probe of the first set of capture probes comprises:
(i) a first spatial barcode; (ii) a first capture domain; and (iii) a first photocleavable cleavage site; and
wherein a first capture probe of the second set of capture probes comprises: (i) a second spatial barcode; (ii) a second capture domain; and (iii) a second photocleavable cleavage site;
wherein the first photocleavable cleavage site of the first capture probe of the first set of capture probes differs from the second photocleavable cleavage site of the first capture probe of the second set of capture probes;
(c) hybridizing the analyte to the first capture domain;
(d) releasing the first set of capture probes from the array; and
(e) determining (i) all or a part of the sequence of the analyte, or a complement thereof, and (ii) the sequence of the first spatial barcode, or a complement thereof, and using the determined sequences of (i) and (ii) to determine the spatial location of the analyte in the biological sample.

127. The method of claim 126, wherein the first photocleavable cleavage site is 5′ to the first spatial barcode and the first capture domain of the first capture probe, and/or wherein and the second photocleavable cleavage site is 5′ to the second spatial barcode and the second capture domain of the first capture probe of the second set of capture probes.

128. The method of claim 126, wherein the first photocleavable cleavage site and the second photocleavable cleavage site comprise one or more nucleosides comprising a photoreactive group, wherein the photoreactive group is selected from o-Nitrobenzyl (o-NB) derivatives, p-Hydroxyphenacyl, TEEP-OH, Aryl sulfide, and nitroindole.

129. The method of claim 126, wherein the releasing of the first set of capture probes comprises exposing the array to light.

130. The method of claim 129, further comprising releasing the second set of capture probes, wherein the first photocleavable cleavage site and the second photocleavable cleavage site are cleaved by exposure to different wavelengths of light.

131. The method of claim 126, wherein releasing the first set of capture probes from the array is performed after the analyte hybridizes to the first capture domain, or wherein hybridizing the analyte to the first capture domain is performed after releasing the first set of capture probes from the array.

132. The method of claim 126, further comprising hybridizing a second analyte in the biological sample or a second analyte from a second biological sample to the second capture domain.

133. The method of claim 132, wherein hybridizing the second analyte to the second capture domain is performed before releasing the second set of capture probes from the array, or wherein hybridizing the second analyte to the second capture domain is performed after releasing the second set of capture probes from the array.

134. The method of claim 126, wherein the array further comprises one or more additional sets of capture probes and wherein the one or more additional sets of capture probes each have a different release mechanism than the first and second set of capture probes, the method further comprising hybridizing one or more additional analytes in the biological sample or in different biological samples to a capture domain of a capture probe in the one or more additional sets of capture probes, and releasing the one or more additional sets of capture probes from the array.

135. The method of claim 126, wherein the analyte is a nucleic acid, optionally, wherein the nucleic acid is mRNA.

136. The method of claim 126, wherein the first capture probe of the first set of capture probes and/or the first capture probe of the second set of capture probes further comprises one or more functional domains, a unique molecular identifier, a cleavage domain, or a combination thereof.

137. The method of claim 126, wherein the determining step comprises sequencing.

138. The method of claim 126, wherein the biological sample is a tissue section.

139. The method of claim 126, further comprising staining the biological sample and/or imaging the biological sample.

140. The method of claim 126, further comprising extending the first capture probe using the analyte as a template, thereby generating an extended capture probe; and optionally generating a complementary strand to the extended capture probe.

141. The method claim 126, wherein the array comprises a plurality of features, wherein a feature in the plurality of features comprises the first set of capture probes and the second set of capture probes.

142. The method of claim 126, wherein the first spatial barcode and the second spatial barcode comprise the same sequence.

143. The method of claim 126, wherein the first spatial barcode and the second spatial barcode comprise different sequences.

144. The method of claim 126, wherein the first capture domain and the second capture domain comprise different sequences.

145. A method for determining a spatial location of an analyte in a biological sample, the method comprising:
(a) disposing the biological sample onto a first substrate;
(b) hybridizing a first probe and a second probe to the analyte, wherein the first probe and the second probe each comprise a sequence that is substantially complementary to sequences of the analyte, and wherein the second probe comprises a capture probe binding domain;
(c) coupling the first probe and the second probe, thereby generating a ligation product;
(d) providing a second substrate comprising an array, wherein the array comprises a first set of capture probes and a second set of capture probes,
wherein a first capture probe of the first set of capture probes comprises:
(i) a first spatial barcode; (ii) a first capture domain; and (iii) a first photocleavable cleavage site; and
wherein a first capture probe of the second set of capture probes comprises: (i) a second spatial barcode; (ii) a second capture domain; and (iii) a second photocleavable cleavage site;
wherein the first photocleavable site of the first capture probe differs from the second photocleavable site of the second capture probe;
(e) hybridizing the ligation product to the first capture domain of the first capture probe;
(f) releasing the first set of capture probes from the array; and
(g) determining (i) all or a part of the sequence of the analyte, or a complement thereof, and (ii) the sequence of the first spatial barcode, or a complement thereof, and using the determined sequences of (i) and (ii) to determine the spatial location of the analyte in the biological sample.
